SEQUENCE LISTING (7 sequences)

SEQ ID NO: 1 - protein, 574 aa, Artificial Sequence
  RSV F protein A2 full length sequence
MELLILKANAITTILTAVTFCFASGQNITEEFYQSTCSAVSKGYLSALRTGWYTSVITIELSNIKKNKCNGTDAKIKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAVKSTTNIMITTIIIVIIVILLSLIAVGLLLYCKARSTPVTLSKDQLSGINNIAFSN

SEQ ID NO: 2 - protein, 27 aa, Artificial Sequence
  Trimerization domain
GYIPEAPRDGQAYVRKDGEWVLLSTFL

SEQ ID NO: 3 - protein, 4 aa, Artificial Sequence
  Linker
SAIG

SEQ ID NO: 4 - protein, 574 aa, Artificial Sequence
  RSV preF2.1
MELLILKANAITTILTAVTFCFASGQNITEEFYQSTCSAVSKGYLGALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAVKSTTNIMITTIIIVIIVILLSLIAVGLLLYCKARSTPVTLSKDQLSGINNIAFSN

SEQ ID NO: 5 - protein, 574 aa, Artificial Sequence
  RSV preF2.2
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKSDELLHNVNAVKSTTNIMITTIIIVIIVILLSLIAVGLLLYCKARSTPVTLSKDQLSGINNIAFSN

SEQ ID NO: 6 - DNA, 1725 bp, Artificial Sequence
  RSV F pre-F2.1
atggagctgctgatcctgaaggccaacgccatcaccaccatcctgaccgccgtgaccttctgcttcgccagcggccagaacatcaccgaggagttctaccagagcacctgcagcgccgtgagcaagggctacctgggcgccctgagaaccggctggtacaccagcgtgatcaccatcgagctgagcaacatcaaggagatcaagtgcaacggcaccgacgccaaggtgaagctgatcaagcaggagctggacaagtacaagaacgccgtgaccgagctgcagctgctgatgcagagcacccccgccaccaacaacagagccagaagagagctgcccagattcatgaactacaccctgaacaacgccaagaagaccaacgtgaccctgagcaagaagagaaagagaagattcctgggcttcctgctgggcgtgggcagcgccatcgccagcggcgtggccgtgagcaaggtgctgcacctggagggcgaggtgaacaagatcaagagcgccctgctgagcaccaacaaggccgtggtgagcctgagcaacggcgtgagcgtgctgaccagcaaggtgctggacctgaagaactacatcgacaagcagctgctgcccatcgtgaacaagcagagctgcagcatccccaacatcgagaccgtgatcgagttccagcagaagaacaacagactgctggagatcaccagagagttcagcgtgaacgccggcgtgaccacccccgtgagcacctacatgctgaccaacagcgagctgctgagcctgatcaacgacatgcccatcaccaacgaccagaagaagctgatgagcaacaacgtgcagatcgtgagacagcagagctacagcatcatgagcatcatcaaggaggaggtgctggcctacgtggtgcagctgcccctgtacggcgtgatcgacaccccctgctggaagctgcacaccagccccctgtgcaccaccaacaccaaggagggcagcaacatctgcctgaccagaaccgacagaggctggtactgcgacaacgccggcagcgtgagcttcttcccccaggccgagacctgcaaggtgcagagcaacagagtgttctgcgacaccatgaacagcctgaccctgcccagcgaggtgaacctgtgcaacgtggacatcttcaaccccaagtacgactgcaagatcatgaccagcaagaccgacgtgagcagcagcgtgatcaccagcctgggcgccatcgtgagctgctacggcaagaccaagtgcaccgccagcaacaagaacagaggcatcatcaagaccttcagcaacggctgcgactacgtgagcaacaagggcgtggacaccgtgagcgtgggcaacaccctgtactacgtgaacaagcaggagggcaagagcctgtacgtgaagggcgagcccatcatcaacttctacgaccccctggtgttccccagcgacgagttcgacgccagcatcagccaggtgaacgagaagatcaaccagagcctggccttcatcagaaagagcgacgagctgctgcacaacgtgaacgccgtgaagagcaccaccaacatcatgatcaccaccatcatcatcgtgatcatcgtgatcctgctgagcctgatcgccgtgggcctgctgctgtactgcaaggccagaagcacccccgtgaccctgagcaaggaccagctgagcggcatcaacaacatcgccttcagcaactga

SEQ ID NO: 7 - DNA, 1725 bp, Artificial Sequence
  RSV F pre-F2.2
atggagctgctgatcctgaaggccaacgccatcaccaccatcctgaccgccgtgaccttctgcttcgccagcggccagaacatcaccgaggagttctaccagagcacctgcagcgccgtgagcaagggctacctgagcgccctgagaaccggctggtacaccagcgtgatcaccatcgagctgagcaacatcaaggagatcaagtgcaacggcaccgacgccaaggtgaagctgatcaagcaggagctggacaagtacaagaacgccgtgaccgagctgcagctgctgatgcagagcacccccgccaccaacaacagagccagaagagagctgcccagattcatgaactacaccctgaacaacgccaagaagaccaacgtgaccctgagcaagaagagaaagagaagattcctgggcttcctgctgggcgtgggcagcgccatcgccagcggcgtggccgtgagcaaggtgctgcacctggagggcgaggtgaacaagatcaagagcgccctgctgagcaccaacaaggccgtggtgagcctgagcaacggcgtgagcgtgctgaccagcaaggtgctggacctgaagaactacatcgacaagcagctgctgcccatcgtgaacaagcagagctgcagcatccccaacatcgagaccgtgatcgagttccagcagaagaacaacagactgctggagatcaccagagagttcagcgtgaacgccggcgtgaccacccccgtgagcacctacatgctgaccaacagcgagctgctgagcctgatcaacgacatgcccatcaccaacgaccagaagaagctgatgagcaacaacgtgcagatcgtgagacagcagagctacagcatcatgagcatcatcaaggaggaggtgctggcctacgtggtgcagctgcccctgtacggcgtgatcgacaccccctgctggaagctgcacaccagccccctgtgcaccaccaacaccaaggagggcagcaacatctgcctgaccagaaccgacagaggctggtactgcgacaacgccggcagcgtgagcttcttcccccaggccgagacctgcaaggtgcagagcaacagagtgttctgcgacaccatgaacagcctgaccctgcccagcgaggtgaacctgtgcaacgtggacatcttcaaccccaagtacgactgcaagatcatgaccagcaagaccgacgtgagcagcagcgtgatcaccagcctgggcgccatcgtgagctgctacggcaagaccaagtgcaccgccagcaacaagaacagaggcatcatcaagaccttcagcaacggctgcgactacgtgagcaacaagggcgtggacaccgtgagcgtgggcaacaccctgtactacgtgaacaagcaggagggcaagagcctgtacgtgaagggcgagcccatcatcaacttctacgaccccctggtgttccccagcaacgagttcgacgccagcatcagccaggtgaacgagaagatcaaccagagcctggccttcatcagaaagagcgacgagctgctgcacaacgtgaacgccgtgaagagcaccaccaacatcatgatcaccaccatcatcatcgtgatcatcgtgatcctgctgagcctgatcgccgtgggcctgctgctgtactgcaaggccagaagcacccccgtgaccctgagcaaggaccagctgagcggcatcaacaacatcgccttcagcaactga

(19) United States
(12) Patent Application Publication
(10) Pub. No.: US 2022/0193219 A1
(43) Pub. Date: Jun. 23, 2022

(54) PROPHYLACTIC TREATMENT OF RESPIRATORY SYNCYTIAL VIRUS INFECTION WITH AN ADENOVIRUS BASED VACCINE

(71) Applicant: JANSSEN VACCINES & PREVENTION B.V., Leiden (NL)

(72) Inventors: Benoit Christophe Stephan CALLENDRET, Den Haag (NL); Jerald C. SADOFF, Washington, DC (US); Els DE PAEPE, Lokeren (BE)

(21) Appl. No.: 17/594,394

(22) PCT Filed: May 14, 2020
(86) PCT No.: PCT/EP2020/063408

§ 371 Date: Oct. 14, 2021

Related U.S. Application Data
(60) Provisional application No. 62/848,186, filed on May 15, 2019.

(51) Int. Cl. A61K 39/12; A61P 31/14; C12N 7/00

(52) U.S. Cl. CPC A61K 39/12; C12N 7/00; A61P 31/14

(57) ABSTRACT

Methods of inducing a protective immune response against respiratory syncytial virus (RSV) and methods of preventing infection and/or replication of RSV, without inducing a severe adverse event in human subjects are described. The methods include administering to the subjects an effective amount of an adenoviral vector encoding a recombinant RSV F polypeptide that is stabilized in a pre-fusion conformation.

### FIELD OF THE INVENTION

The present invention is in the field of medicine. In particular, embodiments of the invention relate to adenovirus-based vaccines and uses thereof for prophylactic treatment of Respiratory Syncytial Virus (RSV) infection.

### BACKGROUND

Respiratory syncytial virus (RSV) is considered to be the most important cause of serious acute respiratory illness in infants and children under 5 years of age (Hall, et al., N Engl J Med. 2009:360; 588-598; Shay et al., JAMA. 1999:282; 1440-1446; Stockman et al., Pediatr Infect Dis J. 2012:31; 5-9). Globally, RSV is responsible for an estimated 3.4 million hospitalizations annually. In the United States, RSV infection in children under 5 years of age is the cause of 57,000 to 175,000 hospitalizations, 500,000 emergency room visits, and approximately 500 deaths each year (Paramore et al., Pharmacoeconomics. 2004:22; 275-284; Shay et al., JAMA. 1999:282; 1440-1446; Stockman et al., Pediatr Infect Dis J. 2012:31; 5-9). In the US, 60% of infants are infected upon initial exposure to RSV (Glezen et al., Am J Dis Child. 1986:140; 543-546), and nearly all children will have been infected with the virus by 2-3 years of age. Immunity to RSV is transient, and repeated infection occurs throughout life (Hall et al., J Infect Dis. 1991:163; 693-698). In children under 1 year of age, RSV is the most important cause of bronchiolitis, and RSV hospitalization is highest among children under 6 months of age (Centers for Disease Control and Prevention (CDC). Respiratory Syncytial Virus Infection (RSV)—Infection and Incidence. Available at: http://www.cdc.gov/rsv/about/infection.html (last accessed 2 Jun. 2016); Hall, et al., N Engl J Med. 2009:360; 588-598). Almost all RSV-related deaths (99%) in children under 5 years of age occur in the developing world (Nair et al., Lancet. 2010:375; 1545-1555). Nevertheless, the disease burden due to RSV in developed countries is substantial, with RSV infection during childhood linked to the development of wheezing, airway hyperreactivity and asthma (Peebles et al., J Allergy Clin Immunol. 2004:113; S15-18; Regnier and Huels, Pediatr Infect Dis J. 2013:32; 820-826; Sigurs et al., Am J Respir Crit Care Med. 2005:171; 137-141; Simoes et al., J Allergy Clin Immunol. 2010:126; 256-262; Simoes et al., J Pediatr. 2007:151; 34-42, 42 e31).

In addition to children, RSV is an important cause of respiratory infections in the elderly, immunocompromised, and those with underlying chronic cardio-pulmonary conditions (Falsey et al., N Engl J Med. 2005:352; 1749-1759). In long-term care facilities, RSV is estimated to infect 5-10% of the residents per year with significant rates of pneumonia (10 to 20%) and death (2 to 5%) (Falsey et al., Clin Microbiol Rev. 2000:13; 371-384). In one epidemiology study of RSV burden, it was estimated that 11,000 elderly persons die annually of RSV in the US (Thompson et al., JAMA. 2003:289; 179-186). These data support the importance of developing an effective vaccine for certain adult populations.

Prophylaxis through passive immunization with a neutralizing monoclonal antibody against the RSV fusion (F) glycoprotein (Synagis® [palivizumab]) is available, but only indicated for premature infants (less than 29 weeks gestational age), children with severe cardio-pulmonary disease or those that are profoundly immunocompromised (American Academy of Pediatrics Committee on Infectious Diseases, American Academy of Pediatrics Bronchiolitis Guidelines Committee. Updated guidance for palivizumab prophylaxis among infants and young children at increased risk of hospitalization for respiratory syncytial virus infection. Pediatrics. 2014:134; 415-420). Synagis has been shown to reduce the risk of hospitalization by 55% (Prevention. Prevention of respiratory syncytial virus infections: indications for the use of palivizumab and update on the use of RSV-IGIV. American Academy of Pediatrics Committee on Infectious Diseases and Committee of Fetus and Newborn. Pediatrics. 1998:102; 1211-1216).

Despite the high disease burden and a strong interest in RSV vaccine development, no licensed vaccine is available for RSV. In the late 1960s, a series of studies were initiated to evaluate a formalin-inactivated RSV vaccine (FI-RSV) adjuvanted with alum, and the results of these studies had a major impact on the RSV vaccine field. Four studies were performed in parallel in children of different age groups with an FI-RSV vaccine delivered by intramuscular injection (Chin et al., Am J Epidemiol. 1969:89; 449-463; Fulginiti et al., Am J Epidemiol. 1969:89; 435-448; Kapikian et al., Am J Epidemiol. 1969:89; 405-421; Kim et al., Am J Epidemiol. 1969:89; 422-434). Eighty percent of the RSV-infected FI-RSV recipients required hospitalization and two children died during the next winter season (Chin et al., Am J Epidemiol. 1969:89; 449-463). Only 5% of the children in the RSV-infected control group required hospitalization. The mechanisms of the observed enhanced respiratory disease (ERD) among the FI-RSV recipients upon reinfection have been investigated and are believed to be the result of an aberrant immune response in the context of small bronchi present in that age group. Data obtained from analysis of patient samples and animal models suggest that FI-RSV ERD is characterized by low neutralizing antibody titers, the presence of low avidity non-neutralizing antibodies promoting immune complex deposition in the airways, reduced cytotoxic CD8+ T-cell priming shown to be important for viral clearance, and enhanced CD4+ T helper type 2 (Th2)-skewed responses with evidence of eosinophilia (Beeler et al., Microb Pathog. 2013:55; 9-15; Connors et al., J Virol. 1992:66; 7444-7451; De Swart et al., J Virol. 2002:76; 11561-11569; Graham et al., J Immunol. 1993:151; 2032-2040; Kim et al., Pediatr Res. 1976:10; 75-78; Murphy et al., J Clin Microbiol. 1986:24; 197-202; Murphy et al., J Clin Microbiol. 1988:26; 1595-1597; Polack et al., J Exp Med. 2002:196; 859-865). It is believed that the chemical interaction of formalin and RSV protein antigens may be one of the mechanisms by which the FI-RSV vaccine promoted ERD upon subsequent RSV infection (Moghaddam et al., Nat Med. 2006:12; 905-907). For these reasons, formalin is no longer used in RSV vaccine development.

In addition to the FI-RSV vaccine, several live-attenuated and subunit RSV vaccines have been examined in animal models and human studies, but many have been inhibited by the inability to achieve the right balance of safety and immunogenicity/efficacy. Live-attenuated vaccines have been specifically challenged by difficulties related to over- and under-attenuation in infants (Belshe et al., J lnfect Dis. 2004:190; 2096-2103; Karron et al., J Infect Dis. 2005:191; 1093-1104; Luongo et al., Vaccine. 2009:27; 5667-5676). With regard to subunit vaccines, the RSV fusion (F) and glycoprotein (G) proteins, which are both membrane proteins, are the only RSV proteins that induce neutralizing antibodies (Shay et al., JAMA. 1999:282; 1440-1446). Unlike the RSV G protein, the F protein is conserved between RSV strains. A variety of RSV F-subunit vaccines have been developed based on the known superior immunogenicity, protective immunity and the high degree of conservation of the F protein between RSV strains (Graham, Immunol Rev. 2011:239; 149-166). The proof-of-concept provided by the currently available anti-F protein neutralizing monoclonal antibody prophylaxis provides support for the idea that a vaccine inducing high levels of long-lasting neutralizing antibody may prevent RSV disease (Feltes et al., Pediatr Res. 2011:70; 186-191; Groothuis et al., J lnfect Dis. 1998:177; 467-469; Groothuis et al., N Engl J Med. 1993:329; 1524-1530). Several studies have suggested that decreased protection against RSV in elderly could be attributed to an age-related decline in interferon gamma (IFNγ) production by peripheral blood mononuclear cells (PBMCs), a reduced ratio of CD8+ to CD4+ T cells, and reduced numbers of circulating RSV-specific CD8+ memory T cells (De Bree et al., J lnfect Dis. 2005:191; 1710-1718; Lee et al., Mech Ageing Dev. 2005:126; 1223-1229; Looney et al., J lnfect Dis. 2002:185; 682-685). High levels of serum neutralizing antibody are associated with less severe infections in elderly (Walsh and Falsey, J Infect Dis. 2004:190; 373-378). It has also been demonstrated that, following RSV infection in adults, serum antibody titers rise rapidly but then slowly return to pre-infection levels after 16 to 20 months (Falsey et al., J Med Virol. 2006:78; 1493-1497). With consideration given to the previously observed ERD in the FI-RSV vaccine studies in the 1960s, future vaccines should promote a strong antigen-specific CD8+ T-cell response and avoid a skewed Th2-type CD4+ T cell response (Graham, Immunol Rev. 2011:239; 149-166).

RSV F protein fuses the viral and host-cell membranes by irreversible protein refolding from the labile pre-fusion conformation to the stable post-fusion conformation. Structures of both conformations have been determined for RSV F (McLellan et al., Science 2013:342, 592-598; McLellan et al., Nat Struct Mol Biol 2010:17, 248-250; McLellan et al., Science 340, 2013:1113-1117; Swanson et al., Proceedings of the National Academy of Sciences of the United States of America 2011:108, 9619-9624), as well as for the fusion proteins from related paramyxoviruses, providing insight into the mechanism of this complex fusion machine. Like other type I fusion proteins, the inactive precursor, RSV F0, requires cleavage during intracellular maturation by a furin-like protease. RSV F0 contains two furin sites (e.g., between amino acid residues 109/110 and 136/137 of the RSV F0 with a GenBank accession No. ACO83301), which leads to three polypeptides: F2, p27 and F1, with the latter containing a hydrophobic fusion peptide (FP) at its N-terminus. To refold from the pre-fusion to the post-fusion conformation, the refolding region 1 (RR1) (e.g., between residue 137 and 216, that includes the FP and heptad repeat A (HRA)) has to transform from an assembly of helices, loops and strands to a long continuous helix. The FP, located at the N-terminal segment of RR1, is then able to extend away from the viral membrane and insert into the proximal membrane of the target cell. Next, the refolding region 2 (RR2), which forms the C-terminal stem in the pre-fusion F spike and includes the heptad repeat B (HRB), relocates to the other side of the RSV F head and binds the HRA coiled-coil trimer with the HRB domain to form the six-helix bundle. The formation of the RR1 coiled-coil and relocation of RR2 to complete the six-helix bundle are the most dramatic structural changes that occur during the refolding process.

Most neutralizing antibodies in human sera are directed against the pre-fusion conformation, but due to its instability the pre-fusion conformation has a propensity to prematurely refold into the post-fusion conformation, both in solution and on the surface of the virions. RSV F polypeptides stabilized in a pre-fusion conformation are described. See, e.g., WO2014/174018, WO2014/202570 and WO 2017/174564. However, there is no report on the safety, efficacy/immunogenicity of such polypeptides in humans. There is a need for a safe and effective vaccine against RSV.

### SUMMARY OF THE INVENTION

In one general aspect, the present application describes a method for inducing a protective immune response against respiratory syncytial virus (RSV) infection in a human subject in need thereof, comprising intramuscularly administering to the subject an effective amount of a pharmaceutical composition, preferably a vaccine, comprising an adenoviral vector comprising a nucleic acid encoding an RSV F polypeptide that is stabilized in a pre-fusion conformation, wherein the effective amount of the pharmaceutical composition comprises about 1×10¹⁰ to about 1×10¹² viral particles of the adenoviral vector per dose.

In certain embodiments, the adenoviral vector is replication-incompetent and has a deletion in at least one of the adenoviral early region 1 (E1 region) and the early region 3 (E3 region).

In certain embodiments, the adenoviral vector is a replication-incompetent Ad26 adenoviral vector having a deletion of the E1 region and the E3 region.

In certain embodiments, the adenoviral vector is a replication-incompetent Ad35 adenoviral vector having a deletion of the E1 region and the E3 region.

In certain embodiments, the recombinant RSV F polypeptide encoded by the adenoviral vector has the amino acid sequence of SEQ ID NO: 4 or SEQ ID NO: 5.

In certain embodiments, the nucleic acid encoding the RSV F polypeptide comprises the polynucleotide sequence of SEQ ID NO: 6 or SEQ ID NO: 7.

In certain embodiments, the effective amount of the pharmaceutical composition comprises about 1×10¹¹ viral particles of the adenoviral vector per dose.

In certain embodiments, the method further comprises administering to the subject an effective amount of the pharmaceutical composition comprising about 1×10¹⁰ to about 1×10¹² viral particles of the adenoviral vector per dose after the initial administration.

In certain embodiments, the subject is susceptible to the RSV infection.

In certain embodiments, the protective immune response is characterized by an absent or reduced RSV viral load in the nasal track and/or lungs of the subject upon exposure to RSV.

In certain embodiments, the protective immune response is characterized by an absent or reduced RSV clinical symptom in the subject upon exposure to RSV.

In certain embodiments, the protective immune response is characterized by neutralizing antibodies to RSV and/or protective immunity against RSV.

In certain embodiments, the administration does not induce any severe adverse event.

The invention also relates to methods for preventing infection and/or replication of RSV without inducing a severe adverse effect in a human subject in need thereof, comprising prophylactically administering intramuscularly to the subject an effective amount of a pharmaceutical composition, preferably a vaccine, comprising about 1×10¹⁰ to about 1×10¹² viral particles per dose of an adenoviral vector comprising a nucleic acid encoding an RSV F polypeptide having the amino acid sequence of SEQ ID NO: 4 or SEQ ID NO: 5, wherein the adenoviral vector is replication-incompetent.

In certain embodiments, the adenoviral vector is a replication-incompetent Ad26 adenoviral vector having a deletion of the E1 region and the E3 region.

In certain embodiments, the nucleic acid encoding the RSV F polypeptide comprises the polynucleotide sequence of SEQ ID NO: 6 or SEQ ID NO: 7.

In certain embodiments, the effective amount of the pharmaceutical composition comprises about 1×10¹¹ viral particles of the adenoviral vector per dose.

In certain embodiments, the method further comprises administering to the subject an effective amount of the pharmaceutical composition comprising about 1×10¹⁰ to about 1×10¹² viral particles of the adenoviral vector per dose after the initial administration.

In certain embodiments, the subject is susceptible to the RSV infection.

In certain embodiments, the protective immune response is characterized by an absent or reduced RSV viral load in the nasal track and/or lungs of the subject upon exposure to RSV.

In certain embodiments, the protective immune response is characterized by an absent or reduced RSV clinical symptom in the subject upon exposure to RSV.

In certain embodiments, the protective immune response is characterized by neutralizing antibodies to RSV and/or protective immunity against RSV.

### BRIEF DESCRIPTION OF THE FIGURES

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows boxplots of AUC Viral Load determined by quantitative RT-PCR of nasal wash samples for the Intent-to-Treat-Challenge Set, with p-value calculated by the Exact Wilcoxon Rank Sum test;

FIG. 2 shows the viral load determined by quantitative RT-PCR of nasal wash samples over time for the Intent-to-Treat-Challenge Set, with the mean +/−SE shown;

FIG. 3 shows boxplots of the peak viral load determined by quantitative RT-PCR of nasal wash samples for the Intent-to-Treat-Challenge Set, with p-value calculated by the Exact Wilcoxon Rank Sum test;

FIG. 4 shows the viral load determined by quantitative culture of RSV of nasal wash samples over time for the Intent-to-Treat-Challenge Set, with the mean +/−SE shown;

FIG. 5 shows boxplots of AUC Viral Load determined by quantitative culture of RSV of nasal wash samples for the Intent-to-Treat-Challenge Set, with p-value calculated by the Exact Wilcoxon Rank Sum test;

FIG. 6 shows the total clinical symptoms scores over time for the Intent-to-Treat-Challenge Set, with the mean +/−SE shown;

FIG. 7 shows boxplots of the AUC of total clinical symptoms scores for the Intent-to-Treat-Challenge Set, with p-value calculated by the Exact Wilcoxon Rank Sum test;

FIG. 8 shows Forest plots of the percentage of subjects with symptomatic RSV infection and of the mean difference (with corresponding 95% CI) between Ad26.RSV.preF and Placebo, for the two RSV infection definitions for the Intent-to-Treat-Challenge Set with the difference in % infected calculated by the Wilson score method;

FIG. 9 shows boxplots of AUC VL determined by quantitative RT-PCR of nasal wash samples, grouped by symptomatic RSV infection definition for the Intent-to-Treat-Challenge Set;

FIG. 10 shows boxplots of AUC VL determined by quantitative culture of RSV of nasal wash samples, grouped by symptomatic RSV infection definition for the Intent-to-Treat-Challenge Set;

FIG. 11 shows boxplots of AUC of total clinical symptoms scores, grouped by symptomatic RSV infection definition for the Intent-to-Treat-Challenge Set;

FIG. 12 shows the weight of mucus produced over time for the Intent-to-Treat-Challenge Set;

FIG. 13 shows the number of tissues used over time for the Intent-to-Treat-Challenge Set;

FIG. 14 shows boxplots of AUC of the weight of mucus produced from baseline to discharge for the Intent-to-Treat-Challenge Set, with p-value calculated by the Exact Wilcoxon Rank Sum test;

FIG. 15 shows the Pre-F IgG serum antibody response, assessed by ELISA, over time for the Per-protocol Immunogenicity Set, with Geometric mean titers with 95% CI shown, and with N representing the number of subjects with data at baseline;

FIG. 16 shows titers of neutralizing antibodies to RSV A2 strain over time for the Per-protocol Immunogenicity Set, with Geometric mean titers with 95% CI shown, and with N representing the number of subjects with data at baseline;

FIG. 17 shows a scatterplot of AUC Viral Load determined by quantitative RT-PCR of nasal wash samples versus titers of Neutralizing Antibodies to RSV A2 strain for the Intent-to-Treat-Challenge Set;

FIG. 18 shows the Pre-F IgG serum antibody response, assessed by ELISA, 28 days post vaccination, grouped by symptomatic RSV infection definition for the Per-protocol Immunogenicity Set; and

FIG. 19 shows titers of neutralizing antibodies to RSV A2 strain 28 days post vaccination, grouped by symptomatic RSV infection definition for the Per-protocol Immunogenicity Set.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The present invention provides methods for inducing a protective immune response against respiratory syncytial virus (RSV) infection in a human subject in need thereof, comprising intramuscularly administering to the subject an effective amount of a pharmaceutical composition, preferably a vaccine, comprising an adenoviral vector comprising a nucleic acid encoding an RSV F polypeptide that is stabilized in a pre-fusion conformation.

As used herein, the term “RSV fusion protein,” “RSV F protein,” “RSV fusion polypeptide” or “RSV F polypeptide” refers to a fusion (F) protein of any group, subgroup, isolate, type, or strain of respiratory syncytial virus (RSV). RSV exists as a single serotype having two antigenic subgroups, A and B. Examples of RSV F protein include, but are not limited to, RSV F from RSV A, e.g. RSV A1 F protein and RSV A2 F protein, and RSV F from RSV B, e.g. RSV B1 F protein and RSV B2 F protein. As used herein, the term “RSV F protein” includes proteins comprising mutations, e.g., point mutations, fragments, insertions, deletions and splice variants of full length wild type RSV F protein.

According to particular embodiments, the RSV F polypeptides that are stabilized in the pre-fusion conformation are derived from an RSV A strain. In certain embodiments the RSV F polypeptides are derived from the RSV A2 strain. RSV F polypeptides that are stabilized in the pre-fusion conformation that are useful in the invention are RSV F proteins having at least one mutation as compared to a wild type RSV F protein, in particular as compared to the RSV F protein having the amino acid sequence of SEQ ID NO: 1. According to particular embodiments, RSV F polypeptides that are stabilized in the pre-fusion conformation that are useful in the invention comprise at least one mutation selected from the group consisting of K66E, N671, I76V, S215P, K394R, S398L, D486N, D489N, and D489Y.

According to particular embodiments, the RSV F polypeptides that are stabilized in the pre-fusion conformation comprise at least one epitope that is recognized by a pre-fusion specific monoclonal antibody, e.g. CR9501. CR9501 comprises the binding regions of the antibodies referred to as 58C5 in WO2011/020079 and WO2012/006596, which binds specifically to RSV F protein in its pre-fusion conformation and not to the post-fusion conformation.

In particular embodiments, the RSV F polypeptides further comprise a heterologous trimerization domain linked to a truncated F1 domain, as described in WO2014/174018 and WO2014/202570. As used herein a “truncated” F1 domain refers to a F1 domain that is not a full length F1 domain, i.e. wherein either N-terminally or C-terminally one or more amino acid residues have been deleted. According to particular embodiments, at least the transmembrane domain and cytoplasmic tail are deleted to permit expression as a soluble ectodomain. In certain embodiments, the trimerization domain comprises SEQ ID NO: 2 and is linked to amino acid residue 513 of the RSV F1 domain, either directly or through a linker. In certain embodiments, the linker comprises the amino acid sequence SAIG (SEQ ID NO: 3).

Examples of RSV F proteins stabilized in a pre-fusion conformation include, but are not limited to those described in WO2014/174018, WO2014/202570 and WO 2017/174564, the contents of which are incorporated herein by reference.

According to particular embodiments, the RSV F protein comprises an amino acid sequence of SEQ ID NO: 4 or SEQ ID NO: 5, or an amino acid sequence that is at least 75%, 80%, 95%, 90% or 95% identical to the amino acid sequence of SEQ ID NO: 4 or SEQ ID NO: 5.

Examples of nucleic acid encoding RSV F protein stabilized in a pre-fusion conformation include SEQ ID NO: 6 and SEQ ID NO: 7. It is understood by a skilled person that numerous different nucleic acid molecules can encode the same polypeptide as a result of the degeneracy of the genetic code. It is also understood that skilled persons can, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleic acid molecule encoding an amino acid sequence” includes all nucleotide sequences that are degenerate versions of each other and that encode the same amino acid sequence. Nucleotide sequences that encode proteins and RNA can include introns. Sequences herein are provided from 5′ to 3′ direction, as custom in the art.

As used herein, the term “vaccine” refers to a composition containing an active component effective to induce a certain degree of immunity in a subject against a certain pathogen or disease, which will result in at least a decrease, and up to complete absence, of the severity, duration or other manifestation of symptoms associated with infection by the pathogen or the disease. In the present invention, the vaccine comprises an adenovirus comprising a nucleic acid encoding an RSV F polypeptide that is stabilized in the pre-fusion conformation. According to embodiments of the application, the vaccine can be used to prevent serious lower respiratory tract disease leading to hospitalization and decrease the frequency of complications such as pneumonia and bronchiolitis due to RSV infection and replication in a subject. In certain embodiments, the vaccine can be a combination vaccine that further comprises other components that induce a protective immune response, e.g. against other proteins of RSV and/or against other infectious agents. The administration of further active components can for instance be done by separate administration or by administering combination products of the vaccines of the invention and the further active components

As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all. Usually, a subject having a “protective immune response” or “protective immunity” against a certain agent will not die as a result of the infection with the agent.

As used herein, the term “induce” and variations thereof refers to any measurable increase in cellular activity. Induction of a protective immune response can include, for example, activation, proliferation, or maturation of a population of immune cells, increasing the production of a cytokine, and/or another indicator of increased immune function. In certain embodiments, induction of an immune response can include increasing the proliferation of B cells, producing antigen-specific antibodies, increasing the proliferation of antigen-specific T cells, improving dendritic cell antigen presentation and/or an increasing expression of certain cytokines, chemokines and co-stimulatory markers.

The ability to induce a protective immune response against RSV F protein can be evaluated either in vitro or in vivo using a variety of assays which are standard in the art. For a general description of techniques available to evaluate the onset and activation of an immune response, see for example Coligan et al. (1992 and 1994, Current Protocols in Immunology; ed. J Wiley & Sons Inc, National Institute of Health). Measurement of cellular immunity can be performed by methods readily known in the art, e.g., by measurement of cytokine profiles secreted by activated effector cells including those derived from CD4+ and CD8+ T-cells (e.g. quantification of IL-4 or IFN gamma-producing cells by ELISPOT), by measuring PBMC proliferation, by measuring NK cell activity, by determination of the activation status of immune effector cells (e.g. T-cell proliferation assays by a classical [3H] thymidine uptake), by assaying for antigen-specific T lymphocytes in a sensitized subject (e.g. peptide-specific lysis in a cytotoxicity assay, etc.). Additionally, IgG and IgA antibody secreting cells with homing markers for local sites which can indicate trafficking to the gut, lung and nasal tissues can be measured in the blood at various times after immunization as an indication of local immunity, and IgG and IgA antibodies in nasal secretions can be measured; Fc function of antibodies and measurement of antibody interactions with cells such as PMNs, macrophages, and NK cells or with the complement system can be characterized; and single cell RNA sequencing analysis can be used to analyze B cell and T cell repertoires.

The ability to induce a protective immune response against RSV F protein can be determined by testing a biological sample (e.g., nasal wash, blood, plasma, serum, PBMCs, urine, saliva, feces, cerebral spinal fluid, bronchoalveolar lavage or lymph fluid) from the subject for the presence of antibodies, e.g. IgG or IgM antibodies, directed to the RSV F protein(s) administered in the composition, e.g. viral neutralizing antibody against RSV A2 (VNA A2), VNA RSV A Memphis 37b, RSV B, pre-F antibodies, post-F antibodies (see for example Harlow, 1989, Antibodies, Cold Spring Harbor Press). For example, titers of antibodies produced in response to administration of a composition providing an immunogen can be measured by enzyme-linked immunosorbent assay (ELISA), other ELISA-based assays (e.g., MSD-Meso Scale Discovery), dot blots, SDS-PAGE gels, ELISPOT, measurement of Fc interactions with complement, PMNs, macrophages and NK cells, with and without complement enhancement, or Antibody-Dependent Cellular Phagocytosis (ADCP) Assay. Exemplary methods are described in Example 1. According to particular embodiments, the induced immune response is characterized by neutralizing antibodies to RSV and/or protective immunity against RSV.

According to particular embodiments, the protective immune response is characterized by the presence of neutralizing antibodies to RSV and/or protective immunity against RSV, preferably detected 8 to 35 days after administration of the pharmaceutical composition, such as 14, 15, 16, 17, 18, 19, 20, 21, 22, 23, 24, 25, 26, 27, 28, 29, 30, 31, 32, 33, 34 or 35 days after administration of the pharmaceutical composition.

According to particular embodiments, the protective immune response is characterized by absent or reduced RSV viral load in the nasal track and/or lungs of the subject, and/or by absent or reduced adverse effects of RSV infection upon exposure to RSV, as compared to that in a subject to whom the pharmaceutical composition was not administered, upon exposure to RSV. The ability to prevent or reduce RSV viral load can be determined, e.g., by calculating the area under the viral load-time curve (VL-AUC in log₁₀ copies/ml) of RSV as determined by quantitative RT-PCR assay, or by quantitative culture, of nasal wash samples. Exemplary methods are described in Example 1.

According to particular embodiments, the protective immune response is characterized by an absent or reduced RSV clinical symptom in the subject upon exposure to RSV. RSV clinical symptoms include, for example, upper respiratory symptoms including, e.g., runny nose, stuffy nose, sneezing, sore throat, earache; lower respiratory symptoms including, e.g., cough, shortness of breath, chest tightness, wheezing, sputum production; and systemic symptoms including, e.g., malaise, headache, muscle and/or joint ache, chilliness/feverishness.

As used herein, the term “adverse event” (AE) refers to any untoward medical occurrence in a subject administered a pharmaceutical product and which does not necessarily have a causal relationship with the treatment. According to embodiments of the invention, AEs are rated on a 4-point scale of increasing severity using the following definitions: Mild (Garde 1): no interference with activity; Moderate (Grade 2): some interference with activity, not requiring medical intervention; Severe (Grade 3): prevents daily activity and requires medical intervention; Potentially life-threatening (Grade 4): symptoms causing inability to perform basis self-care functions OR medical or operative intervention indicated to prevent permanent impairment, persistent disability. A “severe adverse event,” “severe AE,” “SAE” can be any AE occurring at any dose that results in any of the following outcomes: death, where death is an outcome, not an event; life-threatening, referring to an event in which the patient is at risk of death at the time of the event; it does not refer to an event which could hypothetically have caused death had it been more severe; inpatient hospitalization, i.e., an unplanned, overnight hospitalization, or prolongation of an existing hospitalization; persistent or significant incapacity or substantial disruption of the ability to conduct normal life functions; congenital anomaly/birth defect; important medical event (as deemed by the investigator) that may jeopardize the patients or may require medical or surgical intervention to prevent one of the other outcomes listed above (e.g. intensive treatment in an emergency room or at home for allergic bronchospasm or blood dyscrasias or convulsions that do not result in hospitalization). Hospitalization is official admission to a hospital. Hospitalization or prolongation of a hospitalization constitutes criteria for an AE to be serious; however, it is not in itself considered an SAE. In the absence of an AE, hospitalization or prolongation of hospitalization is not considered an SAE. This can be the case, in the following situations: the hospitalization or prolongation of hospitalization is needed for a procedure required by the protocol; or the hospitalization or prolongation of hospitalization is a part of a routine procedure followed by the center (e.g. stent removal after surgery). Hospitalization for elective treatment of a pre-existing condition that did not worsen during the study is not considered an AE. Complications that occur during hospitalization are AEs. If a complication prolongs hospitalization, or meets any of the other SAE criteria, then the event is an SAE.

As used herein, the term “effective amount” refers to an amount of an active ingredient or component that elicits the desired biological or medicinal response in a subject. Selection of a particular effective dose can be determined (e.g., via clinical trials) by those skilled in the art based upon the consideration of several factors, including the disease to be treated or prevented, the symptoms involved, the patient's body mass, the patient's immune status and other factors known by the skilled artisan. The precise dose to be employed in the formulation will also depend on the mode of administration, route of administration, target site, physiological state of the patient, other medications administered and the severity of disease. For example, the effective amount of pharmaceutical composition also depends on whether adjuvant is also administered, with higher dosages being required in the absence of adjuvant.

According to embodiments of the application, an effective amount of pharmaceutical composition comprises an amount of pharmaceutical composition that is sufficient to induce a protective immune response against RSV F protein without inducing a severe adverse event. In particular embodiments, an effective amount of pharmaceutical composition comprises from about 1×10¹⁰ to about 1×10¹² viral particles per dose, preferably about 1×10¹¹ viral particles per dose, of an adenoviral vector comprising a nucleic acid encoding an RSV F polypeptide that is stabilized in a pre-fusion conformation.

According to embodiments of the application, an effective amount of pharmaceutical composition comprises about 1×10¹⁰ to about 1×10¹² viral particles per dose, such as about 1×10¹⁰ viral particles per dose, about 2×10¹⁰ viral particles per dose, about 3×10¹⁰ viral particles per dose, about 4×10¹⁰ viral particles per dose, about 5×10¹⁰ viral particles per dose, about 6×10¹⁰ viral particles per dose, about 7×10¹⁰ viral particles per dose, about 8×10¹⁰ viral particles per dose, about 9×10¹⁰ viral particles per dose, about 1×10¹¹ viral particles per dose, about 2×10¹¹ viral particles per dose, about 3×10¹¹ viral particles per dose, about 4×10¹¹ viral particles per dose, about 5×10¹¹ viral particles per dose, about 6×10¹¹ viral particles per dose, about 7×10¹¹ viral particles per dose, about 8×10¹¹ viral particles per dose, about 9×10¹¹ viral particles per dose, or about 1×10¹² viral particles per dose, of an adenoviral vector comprising a nucleic acid encoding an RSV F polypeptide that is stabilized in a pre-fusion conformation. Preferably the recombinant RSV F polypeptide has an amino acid sequence of SEQ ID NO: 4 or SEQ ID NO: 5, and the adenoviral vector is of serotype 26, such as a recombinant Ad26.

According to particular embodiments, the human subject is susceptible to RSV infection. In certain embodiments, a human subject that is susceptible to RSV infection includes, but is not limited to, an elderly human subject, for example a human subject ≥50 years old, ≥60 years old, preferably ≥65 years old; a young human subject, for example a human subject ≤5 years old, ≤1 year old; and/or a human subject that is hospitalized or a human subject that has been treated with an antiviral compound but has shown an inadequate antiviral response. In certain embodiments, a human subject that is susceptible to RSV infections includes, but is not limited to, a human subject with chronic heart disease, chronic lung disease, and/or immunodeficiencies.

According to particular embodiments, the pharmaceutical composition comprises an adenovirus comprising a nucleic acid molecule encoding an RSV F polypeptide that is stabilized in the pre-fusion conformation.

In certain embodiments, the vector is a human recombinant adenovirus, also referred to as recombinant adenoviral vectors. The preparation of recombinant adenoviral vectors is well known in the art. The term “recombinant” for an adenovirus, as used herein implicates that it has been modified by the hand of man, e.g. it has altered terminal ends actively cloned therein and/or it comprises a heterologous gene, i.e. it is not a naturally occurring wild type adenovirus.

In certain embodiments, an adenoviral vector according to the invention is deficient in at least one essential gene function of the E1 region, e.g. the E1a region and/or the E1b region, of the adenoviral genome that is required for viral replication. In certain embodiments, an adenoviral vector according to the invention is deficient in at least part of the non-essential E3 region. In certain embodiments, the vector is deficient in at least one essential gene function of the E1 region and at least part of the non-essential E3 region. The adenoviral vector can be “multiply deficient,” meaning that the adenoviral vector is deficient in one or more essential gene functions in each of two or more regions of the adenoviral genome. For example, the aforementioned E1-deficient or E1-, E3-deficient adenoviral vectors can be further deficient in at least one essential gene of the E4 region and/or at least one essential gene of the E2 region (e.g., the E2A region and/or E2B region).

Adenoviral vectors, methods for construction thereof and methods for propagating thereof, are well known in the art and are described in, for example, U.S. Pat. Nos. 5,559,099, 5,837,511, 5,846,782, 5,851,806, 5,994,106, 5,994,128, 5,965,541, 5,981,225, 6,040,174, 6,020,191, and 6,113,913, and Thomas Shenk, “Adenoviridae and their Replication”, M. S. Horwitz, “Adenoviruses”, Chapters 67 and 68, respectively, in Virology, B. N. Fields et al., eds., 3d ed., Raven Press, Ltd., New York (1996), and other references mentioned herein. Typically, construction of adenoviral vectors involves the use of standard molecular biological techniques, such as those described in, for example, Sambrook et al., Molecular Cloning, a Laboratory Manual, 2d ed., Cold Spring Harbor Press, Cold Spring Harbor, N.Y. (1989), Watson et al., Recombinant DN A, 2d ed., Scientific American Books (1992), and Ausubel et al., Current Protocols in Molecular Biology, Wiley Interscience Publishers, NY (1995), and other references mentioned herein.

In certain embodiments, the adenovirus is a human adenovirus of the serotype 26 or 35.

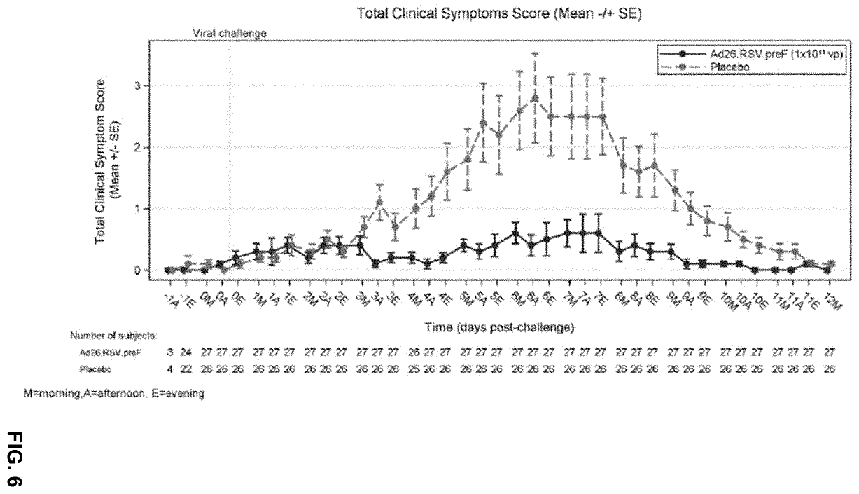

Preparation of rAd26 vectors is described, for example, in WO 2007/104792 and in Abbink et al., Virol. 2007:81(9): 4654-63. Exemplary genome sequences of Ad26 are found in GenBank Accession EF 153474 and in SEQ ID NO: 1 of WO 2007/104792. Preparation of rAd35 vectors is described, for example, in U.S. Pat. No. 7,270,811, in WO 00/70071, and in Vogels et al, J Virol. 2003:77(15): 8263-71. Exemplary genome sequences of Ad35 are found in GenBank Accession AC 000019 and in FIG. 6 of WO 00/70071.

A recombinant adenovirus according to the invention can be replication-competent or replication-deficient. In certain embodiments, the adenovirus is replication deficient, e.g. because it contains a deletion in the E1 region of the genome. As known to the skilled person, in case of deletions of essential regions from the adenovirus genome, the functions encoded by these regions have to be provided in trans, preferably by the producer cell, i.e. when parts or whole of E1, E2 and/or E4 regions are deleted from the adenovirus, these have to be present in the producer cell, for instance integrated in the genome thereof, or in the form of so-called helper adenovirus or helper plasmids. The adenovirus can also have a deletion in the E3 region, which is dispensable for replication, and hence such a deletion does not have to be complemented.

In certain embodiments, the adenovirus is a replication-incompetent adenovirus. According to particular embodiments, the adenovirus is a replication-incompetent Ad26 adenovirus. According to particular embodiments, the adenovirus is a replication-incompetent Ad35 adenovirus.

A producer cell (sometimes also referred to in the art and herein as “packaging cell” or “complementing cell” or “host cell”) that can be used can be any producer cell wherein a desired adenovirus can be propagated. For example, the propagation of recombinant adenovirus vectors is done in producer cells that complement deficiencies in the adenovirus. Such producer cells preferably have in their genome at least an adenovirus E1 sequence, and thereby are capable of complementing recombinant adenoviruses with a deletion in the E1 region. Any E1-complementing producer cell can be used, such as human retina cells immortalized by E1, e.g. 911 or PER.C6 cells (see U.S. Pat. No. 5,994,128), E1-transformed amniocytes (See EP patent 1230354), E1-transformed A549 cells (see e.g. WO 98/39411, U.S. Pat. No. 5,891,690), GH329:HeLa (Gao et al., Human Gene Therapy 2000:11: 213-219), 293, and the like. In certain embodiments, the producer cells are for instance HEK293 cells, or PER.C6 cells, or 911 cells, or IT293SF cells, and the like.

For non-subgroup C E1-deficient adenoviruses such as Ad35 (subgroup B) or Ad26 (subgroup D), it is preferred to exchange the E4-orf6 coding sequence of these non-subgroup C adenoviruses with the E4-orf6 of an adenovirus of subgroup C such as Ad5. This allows propagation of such adenoviruses in well-known complementing cell lines that express the E1 genes of Ad5, such as for example 293 cells or PER.C6 cells (see, e.g. Havenga et al., J Gen. Virol. 2006:87: 2135-2143; WO 03/104467, incorporated in its entirety by reference herein). In certain embodiments, an adenovirus that can be used is a human adenovirus of serotype 35, with a deletion in the E1 region into which the nucleic acid encoding RSV F protein antigen has been cloned, and with an E4 orf6 region of Ad5. In certain embodiments, the adenovirus in the vaccine composition of the invention is a human adenovirus of serotype 26, with a deletion in the E1 region into which the nucleic acid encoding RSV F protein antigen has been cloned, and with an E4 orf6 region of Ad5.

In alternative embodiments, there is no need to place a heterologous E4orf6 region (e.g. of Ad5) in the adenoviral vector, but instead the E1-deficient non-subgroup C vector is propagated in a cell line that expresses both E1 and a compatible E4orf6, e.g. the 293-ORF6 cell line that expresses both E1 and E4orf6 from Ad5 (see e.g. Brough et al, J Virol. 1996:70: 6497-501 describing the generation of the 293-ORF6 cells; Abrahamsen et al, J Virol. 1997:71: 8946-51 and Nan et al, Gene Therapy 2003:10: 326-36 each describing generation of E1 deleted non-subgroup C adenoviral vectors using such a cell line).

Alternatively, a complementing cell that expresses E1 from the serotype that is to be propagated can be used (see e.g. WO 00/70071, WO 02/40665).

For subgroup B adenoviruses, such as Ad35, having a deletion in the E1 region, it is preferred to retain the 3′ end of the E1 B 55K open reading frame in the adenovirus, for instance the 166 bp directly upstream of the pIX open reading frame or a fragment comprising this such as a 243 bp fragment directly upstream of the pIX start codon (marked at the 5 end by a Bsu361 restriction site in the Ad35 genome), since this increases the stability of the adenovirus because the promoter of the pIX gene is partly residing in this area (see, e.g. Havenga et al, 2006, J. Gen. Virol. 87: 2135-2143; WO 2004/001032, incorporated by reference herein).

Recombinant adenovirus can be prepared and propagated in host cells, according to well-known methods, which entail cell culture of the host cells that are infected with the adenovirus. The cell culture can be any type of cell culture, including adherent cell culture, e.g. cells attached to the surface of a culture vessel or to microcarriers, as well as suspension culture.

According to particular embodiments, the pharmaceutical composition further comprises a pharmaceutically acceptable carrier or excipient. As used herein, the term “pharmaceutically acceptable” means that the carrier or excipient, at the dosages and concentrations employed, will not cause any unwanted or harmful effects in the subjects to which they are administered. Such pharmaceutically acceptable carriers and excipients are well known in the art (see Remington's Pharmaceutical Science (15th ed.), Mack Publishing Company, Easton, Pa., 1980). The preferred formulation of the pharmaceutical composition depends on the intended mode of administration and therapeutic application. The compositions can include pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or non-toxic, non-therapeutic, non-immunogenic stabilizers, and the like. It will be understood that the characteristics of the carrier, excipient or diluent will depend on the route of administration for a particular application.

In some embodiments, the pharmaceutically acceptable carrier comprises one or more salts, such as sodium chloride, potassium chloride, magnesium chloride, one or more amino acids, such as arginine, glycine, histidine and/or methionine, one or more carbohydrates, such as lactose, maltose, sucrose, one or more surfactants, such as polysorbate 20, polysorbate 80, one or more chelators, such as ethylenediaminetetracetic acid (EDTA), and ethylenediamine-N,N′-disuccinic acid (EDDS), and one or more alcohols such as ethanol and methanol. Preferably, the pharmaceutical composition has a pH of 5 to 8, such as a pH of 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, or any value in between.

In some embodiments, a pharmaceutical composition for use in the invention comprises sodium chloride, potassium chloride, and/or magnesium chloride at a concentration of 1 mM to 100 mM, 25 mM to 100 mM, 50 mM to 100 mM, or 75 mM to 100 mM. For example, the concentration of sodium chloride, potassium chloride, and/or magnesium chloride can be 1 mM, 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, or any concentration in between.

In some embodiments, a pharmaceutical composition for use in the invention comprises histidine, arginine, and/or glycine at a concentration of 1 mM to 50 mM, 5 mM to 50 mM, 5 mM to 30 mM, 5 mM to 20 mM, or 10 mM to 20 mM. For example, the concentration of histidine, arginine, and/or glycine can be 1 mM, 2 mM 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM or 50 mM, or any concentration in between.

In some embodiments, a pharmaceutical composition for use in the invention comprises sucrose, lactose, and/or maltose at a concentration of 1% to 10% weight by volume (w/v) or 5% to 10% (w/v). For example, the concentration of sucrose, lactose, and/or maltose can be 1% (w/v), 1.5% (w/v), 2% (w/v), 2.5% (w/v), 3% (w/v), 3.5% (w/v), 4% (w/v), 4.5% (w/v), 5% (w/v), 5.5% (w/v), 6% (w/v), 6.5% (w/v), 7% (w/v), 7.5% (w/v), 8% (w/v), 8.5% (w/v), 9% (w/v), 9.5% (w/v), or 10% (w/v), or any concentration in between.

In some embodiments, a pharmaceutical composition for use in the invention comprises polysorbate 20 (PS20) and/or polysorbate 80 (PS80) at a concentration of 0.01% (w/v) to 0.1% (w/v), 0.01% (w/v) to 0.08% (w/v), or 0.02% (w/v) to 0.05% (w/v). For example, the concentration of polysorbate 20 and/or polysorbate 80 can be 0.01%, 0.02%, 0.03%, 0.04%, 0.05%, 0.06%, 0.07%, 0.08%, 0.09% or 0.1% (w/v), or any concentration in between.

In some embodiments, a pharmaceutical composition for use in the invention comprises ethylenediaminetetracetic acid (EDTA) and/or ethylenediamine-N,N′-disuccinic acid (EDDS) at a concentration of 0.1 mM to 5 mM, 0.1 mM to 2.5 mM, or 0.1 to 1 mM. For example, the concentration of EDTA and/or EDDS can be 0.1 mM, 0.2 mM, 0.3 mM, 0.4 mM, 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, or 5 mM, or any concentration in between.

In some embodiments, a pharmaceutical composition for use in the invention comprises ethanol and/or methanol at a concentration of 0.1% to 5% weight by volume (w/v) or 0.5% to 5% (w/v). For example, the concentration of sucrose, lactose, and/or maltose can be 0.1% (w/v), 0.2% (w/v), 0.3% (w/v), 0.4% (w/v), 0.5% (w/v), 0.6% (w/v), 0.7% (w/v), 0.8% (w/v), 0.9% (w/v), 1% (w/v), 1.5% (w/v), 2% (w/v), 2.5% (w/v), 3% (w/v), 3.5% (w/v), 4% (w/v), 4.5% (w/v), or 5% (w/v), or any concentration in between.

Pharmaceutical compositions comprising an adenovirus comprising a nucleic acid molecule encoding an RSV F polypeptide that is stabilized in the pre-fusion conformation for use in the invention can be prepared by any method known in the art in view of the present disclosure. For example, an adenovirus comprising a nucleic acid molecule encoding an RSV F polypeptide that is stabilized in the pre-fusion conformation can be mixed with one or more pharmaceutically acceptable carriers to obtain a solution. The solution can be stored as a frozen liquid at a controlled temperature ranging from −55° C.±10° C. to −85° C.±10° C. in an appropriate vial until administered to the subject.

In certain embodiments, pharmaceutical compositions according to the invention further comprise one or more adjuvants. Adjuvants are known in the art to further increase the immune response to an applied antigenic determinant. The terms “adjuvant” and “immune stimulant” are used interchangeably herein and are defined as one or more substances that cause stimulation of the immune system. In this context, an adjuvant is used to enhance a protective immune response to the RSV F polypeptides of the pharmaceutical compositions of the invention. Examples of suitable adjuvants include aluminium salts such as aluminium hydroxide and/or aluminium phosphate; oil-emulsion compositions (or oil-in-water compositions), including squalene-water emulsions, such as MF59 (see e.g. WO 90/14837); saponin formulations, such as for example QS21 and Immunostimulating Complexes (ISCOMS) (see e.g. U.S. Pat. No. 5,057,540; WO 90/03184, WO 96/11711, WO 2004/004762, WO 2005/002620); bacterial or microbial derivatives, examples of which are monophosphoryl lipid A (MPL), 3-O-deacylated MPL (3dMPL), CpG-motif containing oligonucleotides, ADP-ribosylating bacterial toxins or mutants thereof, such as E. coli heat labile enterotoxin LT, cholera toxin CT, and the like; eukaryotic proteins (e.g. antibodies or fragments thereof (e.g. directed against the antigen itself or CD1a, CD3, CD7, CD80) and ligands to receptors (e.g. CD40L, GMCSF, GCSF, etc.), which stimulate immune response upon interaction with recipient cells. In certain embodiments the pharmaceutical compositions of the invention comprise aluminium as an adjuvant, e.g. in the form of aluminium hydroxide, aluminium phosphate, aluminium potassium phosphate, or combinations thereof, in concentrations of 0.05-5 mg, e.g. 0.075-1.0 mg, of aluminium content per dose.

The pharmaceutical compositions according to the invention can be used e.g. in stand-alone prophylaxis of a disease or condition caused by RSV, or in combination with other prophylactic and/or therapeutic treatments, such as (existing or future) vaccines, antiviral agents and/or monoclonal antibodies.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a pharmaceutical composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

The timing of administrations can vary significantly from once a day, to once a year, to once a decade. A typical regimen consists of an immunization followed by booster injections at time intervals, such as 1 to 24 week intervals. Another regimen consists of an immunization followed by booster injections 1, 2, 4, 6, 8, 10 and 12 months later. Another regimen entails an injection every two months for life. Another regimen entails an injection every year or every 2, 3, 4 or 5 years. Alternatively, booster injections can be on an irregular basis as indicated by monitoring of immune response.

It is readily appreciated by those skilled in the art that the regimen for the priming and boosting administrations can be adjusted based on the measured immune responses after the administrations. For example, the boosting compositions are generally administered weeks or months after administration of the priming composition, for example, about 1 week, or 2-3 weeks or 4 weeks, or 8 weeks, or 16 weeks, or 20 weeks, or 24 weeks, or 28 weeks, or 32 weeks, or 36 weeks, or 40 weeks, or 44 weeks, or 48 weeks, or 52 weeks, or 56 weeks, or 60 weeks, or 64 weeks, or 68 weeks, or 72 weeks, or 76 weeks, or one to two years after administration of the priming composition.

According to particular aspects, one or more boosting immunizations can be administered. The antigens in the respective priming and boosting compositions, however many boosting compositions are employed, need not be identical, but should share antigenic determinants or be substantially similar to each other.

Pharmaceutical compositions of the present invention can be formulated according to methods known in the art in view of the present disclosure.

The pharmaceutical compositions can be administered by suitable means for prophylactic and/or therapeutic treatment. Non-limiting embodiments include parenteral administration, such as intradermal, intramuscular, subcutaneous, transcutaneous, or mucosal administration, e.g. intranasal, oral, and the like. In one embodiment, a composition is administered by intramuscular injection. The skilled person knows the various possibilities to administer a pharmaceutical composition in order to induce an immune response to the antigen(s) in the pharmaceutical composition. In certain embodiments, a composition of the invention is administered intramuscularly.

The invention also provides methods for preventing infection and/or replication of RSV without inducing a severe adverse effect in a human subject in need thereof. In particular embodiments, the method comprises prophylactically administering to the subject an effective amount of a pharmaceutical composition, preferably a vaccine, comprising an adenoviral vector comprising a nucleic acid encoding an RSV F polypeptide that is stabilized in a pre-fusion conformation. This will reduce adverse effects resulting from RSV infection in a subject, and thus contribute to protection of the subject against such adverse effects upon administration of the pharmaceutical composition.

According to particular embodiments, the prevented infection and/or replication of RSV is characterized by absent or reduced RSV viral load in the nasal track and/or lungs of the subject, and/or by absent or reduced symptom of RSV infection upon exposure to RSV, as compared to that in a subject to whom the pharmaceutical composition was not administered, upon exposure to RSV. In certain embodiments, absent RSV viral load or absent adverse effects of RSV infection means reduced to such low levels that they are not clinically relevant.

According to particular embodiments, the prevented infection and/or replication of RSV is characterized by an absent or reduced RSV clinical symptom in the subject upon exposure to RSV.

According to particular embodiments, the prevented infection and/or replication of RSV is characterized by the presence of neutralizing antibodies to RSV and/or protective immunity against RSV, preferably detected 8 to 35 days after administration of the pharmaceutical composition, such as 14, 15, 16, 17, 18, 19, 20, 21, 22, 23, 24, 25, 26, 27, 28, 29, 30, 31, 32, 33, 34 or 35 days after administration of the pharmaceutical composition. More preferably, the neutralizing antibodies against RSV are detected about 6 months to 5 years after the administration of the pharmaceutical composition, such as 6 months, 1 year, 2 years, 3 years, 4 years or 5 years after administration of the pharmaceutical composition.

According to embodiments of the application, an effective amount of pharmaceutical composition comprises an amount of pharmaceutical composition that is sufficient to prevent infection and/or replication of RSV without inducing a severe adverse event. In particular embodiments, an effective amount of pharmaceutical composition comprises from about 1×10¹⁰ to about 1×10¹² viral particles per dose, preferably about 1×10¹¹ viral particles per dose, of an adenoviral vector comprising a nucleic acid encoding an RSV F polypeptide that is stabilized in a pre-fusion conformation.

According to embodiments of the application, an effective amount of pharmaceutical composition comprises about 1×10¹⁰ to about 1×10¹² viral particles per dose, such as about 1×10¹⁰ viral particles per dose, about 2×10¹⁰ viral particles per dose, about 3×10¹⁰ viral particles per dose, about 4×10¹⁰ viral particles per dose, about 5×10¹⁰ viral particles per dose, about 6×10¹⁰ viral particles per dose, about 7×10¹⁰ viral particles per dose, about 8×10¹⁰ viral particles per dose, about 9×10¹⁰ viral particles per dose, about 1×10¹¹ viral particles per dose, about 2×10¹¹ viral particles per dose, about 3×10¹¹ viral particles per dose, about 4×10¹¹ viral particles per dose, about 5×10¹¹ viral particles per dose, about 6×10¹¹ viral particles per dose, about 7×10¹¹ viral particles per dose, about 8×10¹¹ viral particles per dose, about 9×10¹¹ viral particles per dose, or about 1×10¹² viral particles per dose, of an adenoviral vector comprising a nucleic acid encoding an RSV F polypeptide that is stabilized in a pre-fusion conformation. Preferably the recombinant RSV F polypeptide has an amino acid sequence of SEQ ID NO: 4 or SEQ ID NO: 5, and the adenoviral vector is of serotype 26, such as a recombinant Ad26.

The invention also provides methods for vaccinating a subject against RSV infection without inducing a severe adverse effect in a human subject in need thereof. In particular embodiments, the method comprises administering to the subject an effective amount of a pharmaceutical composition comprising an adenoviral vector comprising a nucleic acid encoding an RSV F polypeptide that is stabilized in a pre-fusion conformation.

According to embodiments of the application, an effective amount of pharmaceutical composition comprises an amount of pharmaceutical composition that is sufficient to vaccinate a subject against RSV infection without inducing a severe adverse event. In particular embodiments, an effective amount of pharmaceutical composition comprises from about 1×10¹⁰ to about 1×10¹² viral particles per dose, preferably about 1×10¹¹ viral particles per dose, of an adenoviral vector comprising a nucleic acid encoding an RSV F polypeptide that is stabilized in a pre-fusion conformation.

According to embodiments of the application, an effective amount of pharmaceutical composition comprises about 1×10¹⁰ to about 1×10¹² viral particles per dose, such as about 1×10¹⁰ viral particles per dose, about 2×10¹⁰ viral particles per dose, about 3×10¹⁰ viral particles per dose, about 4×10¹⁰ viral particles per dose, about 5×10¹⁰ viral particles per dose, about 6×10¹⁰ viral particles per dose, about 7×10¹⁰ viral particles per dose, about 8×10¹⁰ viral particles per dose, about 9×10¹⁰ viral particles per dose, about 1×10¹¹ viral particles per dose, about 2×10¹¹ viral particles per dose, about 3×10¹¹ viral particles per dose, about 4×10¹¹ viral particles per dose, about 5×10¹¹ viral particles per dose, about 6×10¹¹ viral particles per dose, about 7×10¹¹ viral particles per dose, about 8×10¹¹ viral particles per dose, about 9×10¹¹ viral particles per dose, or about 1×10¹² viral particles per dose, of an adenoviral vector comprising a nucleic acid encoding an RSV F polypeptide that is stabilized in a pre-fusion conformation. Preferably the recombinant RSV F polypeptide has an amino acid sequence of SEQ ID NO: 4 or SEQ ID NO: 5, and the adenoviral vector is of serotype 26, such as a recombinant Ad26.

### EXAMPLES

The following examples of the invention are to further illustrate the nature of the invention. It should be understood that the following examples do not limit the invention and that the scope of the invention is to be determined by the appended claims.

### Example 1: Phase 2a Human Challenge Study

An exploratory, Phase 2a, randomized, double-blind, placebo-controlled study was carried out to evaluate the prophylactic efficacy of a single intramuscular immunization of Ad26.RSV.preF, a replication-incompetent Ad26 containing a DNA transgene that encodes for a pre-fusion conformation-stabilized F protein (pre-F) of a RSV A2 strain, against Respiratory Syncytial Virus infection in a virus challenge model in healthy 18- to 50-year-old adults.

Study Design/Overview—

A single center, randomized, placebo-controlled, double-blind Phase 2a human challenge study was conducted in at least 44 healthy male and female subjects aged 18-50 years who were pre-screened for susceptibility to RSV infection, i.e., had levels of RSV neutralizing antibodies compatible with susceptibility to RSV infection. A schematic overview of the study design and groups is depicted in Table 1 below:

TABLE 1

| Group | N | Day −28 | Day 0* |
|---|---|---|---|
| Group 1 | 22 | Ad26.RSV.preF | Challenge with RSV-A |
| | | (1 ×10¹¹ vp) | Memphis 37b** |
| Group 2 | 22 | Placebo | |

*ie, not less than 24 or more than 90 days after vaccination.
**Subjects will be challenged in two or more cohorts of up to 22 subjects per cohort. Within each cohort, subjects will be randomized 1:1 to 1 × 10¹¹ vp of Ad26RSV preF or placebo.

Randomization: Subjects were enrolled into two different groups (Ad26.RSV.preF or Placebo), each comprising of at least 22 healthy adult subjects, with a 1:1 randomization ratio.

Vaccination Schedules/Study duration: The study consisted of a screening phase (56 to 3 days prior vaccination), a vaccination phase in which subjects were vaccinated at Day-28 with Ad26.RSV.preF, a replication-incompetent (delta-Early region 1/Early region 3 [E1/E3]) Ad26 vector containing the sequence encoding for the full length F protein of the RSV A2 strain stabilized in a pre-fusion conformation; and a viral challenge phase where subjects entered the quarantine unit and were challenged on Day 0 (24 to 90 days after vaccination) with RSV-A Memphis 37b. 12 days after the challenge, subjects were discharged and followed up to 6 months after the vaccination.

Primary efficacy endpoint: The area under the viral load-time curve (VL-AUC in log₁₀ copies/ml) of RSV as determined by quantitative RT-PCR assay of nasal wash samples was assessed. Nasal wash samples were taken every 12 (±1) hours beginning two days after inoculation of the challenge virus. VL-AUC was calculated based on the viral load values measured twice daily, starting with the baseline value (last available measurement before challenge), and ending with the last available value before discharge.

Major secondary and exploratory endpoints: peak viral load; viral load of RSV-A Memphis 37b as determined by quantitative culture of RSV of nasal wash samples and the corresponding AUC; total clinical symptom score and corresponding AUC over time; total weight of mucus produced and tissue count; proportion of subjects with symptomatic RSV infection; safety and tolerability assessed by solicited AEs, unsolicited AEs, and SAEs; humoral immune responses elicited by Ad26.RSV.preF and to challenge with RSV-A Memphis 37b were all assessed.

Results—

A total of 63 subjects were randomized and vaccinated, 31 subjects in the active group, 32 in Placebo. 4 subjects in the active group and 6 in the placebo group discontinued the study before being challenged (reasons: lost to follow-up (6 subjects), physician decision (3 subjects) and protocol deviation (1 subject)), resulting in 27 challenged subjects in the active group and 26 in the placebo group.

1. Efficacy: The efficacy analysis was based on the Intent-to-Treat-Challenge (ITTc) population, which is defined as all subjects who were randomized, vaccinated and challenged. The ITTc population contained 53 subjects: 27 in the Ad26.RSV.preF group and 26 Placebo subjects. An effect of the primary endpoint that was significant at 5% (one-sided) was considered a significant effect. An effect that was significant at 20% (one-sided) was considered a trend.

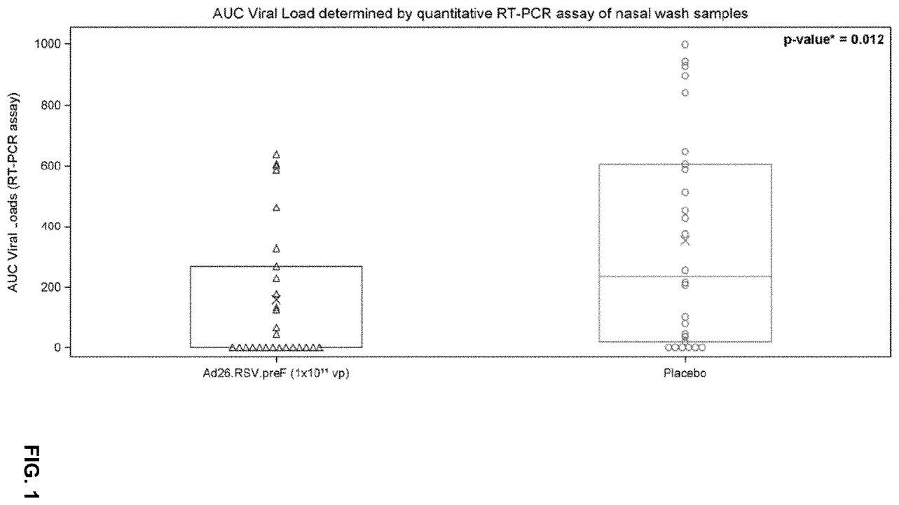

2. Primary efficacy endpoint analysis: The difference in AUC viral loads (VL), determined by RT-PCR of nasal wash samples, between the Ad26.RSV.preF and the Placebo group is summarized in Table 2 and graphically depicted in FIG. 1. The median (Q1; Q3) AUC VL from baseline to discharge was 0 (0; 268.8) for the Ad26.RSV.preF group and 236 (20.3; 605.8) for the Placebo group. The one-sided Exact Wilcoxon Rank Sum test p-value was 0.0012, indicating that there was a significant reduction in VL-AUC in the Ad26.RSV.preF group compared to the Placebo group.

TABLE 2
Primary Efficacy Endpoint: AUC Viral Load determined by
quantitative RT-PCR assay of nasal wash samples; ITTc Set

| AUC Viral Load | | | Difference |
|---|---|---|---|
| from Baseline | | | Ad26.RSV.preF - |
| to Discharge | N | Median (Q1; Q3) | Placebo p-value* |
| Ad26.RSV.preF | 27 | 0 (0.0, 268.8) | 0.012 |
| (1 × 10¹¹ vp) | | | |
| Placebo | 26 | 236 (20.3; 605.8) | |

*Exact Wilcoxon Rank Sum test

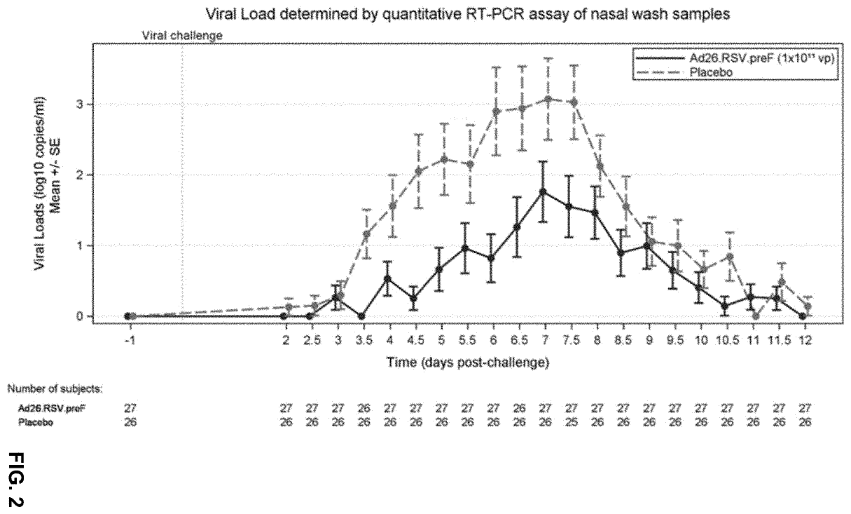

The mean and standard error (SE) of the VLs determined by RT-PCT of nasal wash samples, by day, are graphically depicted in FIG. 2. The peak VL occurred at the first RT-PCR sample collected at Day 7 (morning) in both groups.

3. Secondary and exploratory efficacy endpoint analysis: The study was powered only for the primary efficacy endpoint and not for any of the secondary endpoints. Thus, interpretation of the p-values was done with caution.

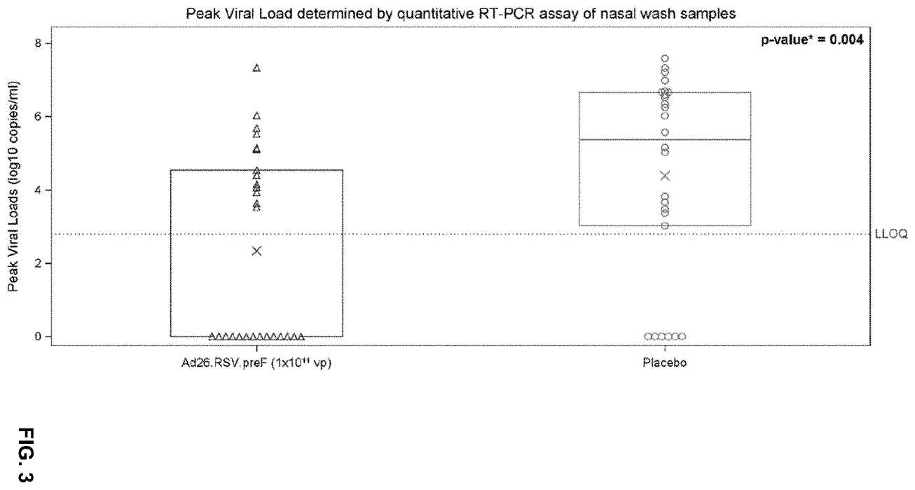

3a. Peak viral load: The difference of peak VL observed during the quarantine of the quantitative RT-PCR assay of the nasal wash samples between the Ad26.RSV.preF and Placebo group is depicted in FIG. 3. The median (Q1; Q3) peak VL was 0 (0; 4.539) log 10 copies/ml for the Ad26.RSV.preF group and 5.365 (3.027; 6.665) log 10 copies/ml for the Placebo group.

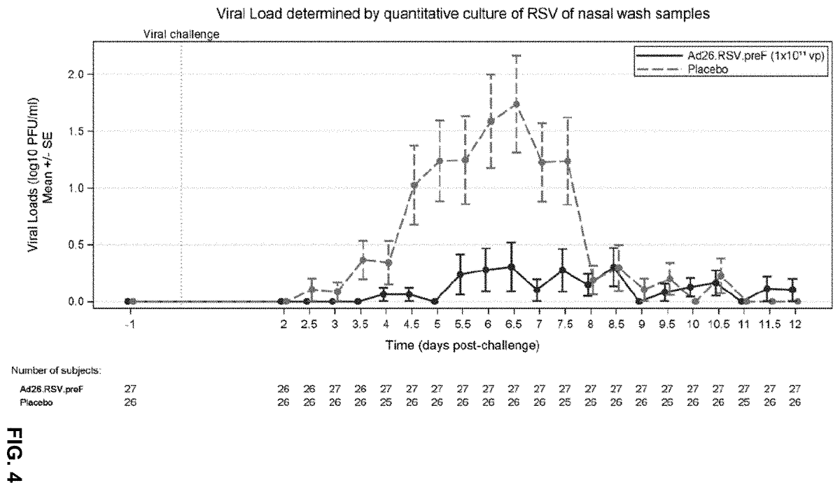

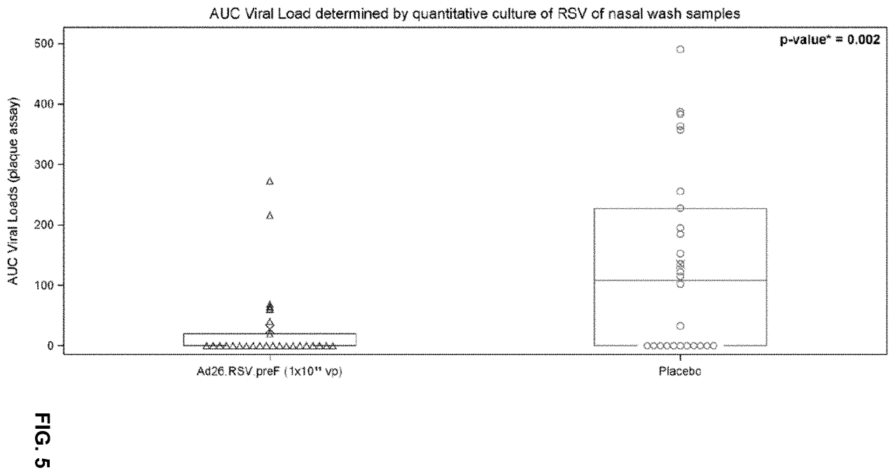

3b. Viral load AUC: The mean and SE of the VLs of RSV-A Memphis 37b determined by quantitative culture of RSV of nasal wash samples, by day, from baseline to discharge, is depicted in FIG. 4. The peak VL for the Placebo group was observed at day 6 in the evening. Boxplots of the AUCs are presented in FIG. 5. The median (Q1; Q3) AUC VL from baseline to discharge was 0 (0; 20.3) for the Ad26.RSV.preF group and 109 (0; 227.5) for the Placebo group.

3c. Total clinical symptoms: 13 self-reportable symptoms were collected in the Subject Symptoms Card three times a day (morning, afternoon and evening). The symptoms were defined as follows:
Upper Respiratory symptoms: runny nose, stuffy nose, sneezing, sore throat, earache
Lower Respiratory symptoms: cough, shortness of breath, chest tightness, wheeze
Systemic symptoms: malaise, headache, muscle and/or joint ache, chilliness/feverishness

The total clinical symptom score was determined as the sum of the scores (grades) of the 13 self-reportable symptoms on the Subject Symptoms Card as follows:
0=‘I have No symptom’
1=‘just noticeable’
2=‘It's clearly bothersome from time to time, but it doesn't stop me from participating in activities’
3=‘It's quite bothersome most or all the time and it stops me from participating in activities’
4=‘Symptoms at rest’

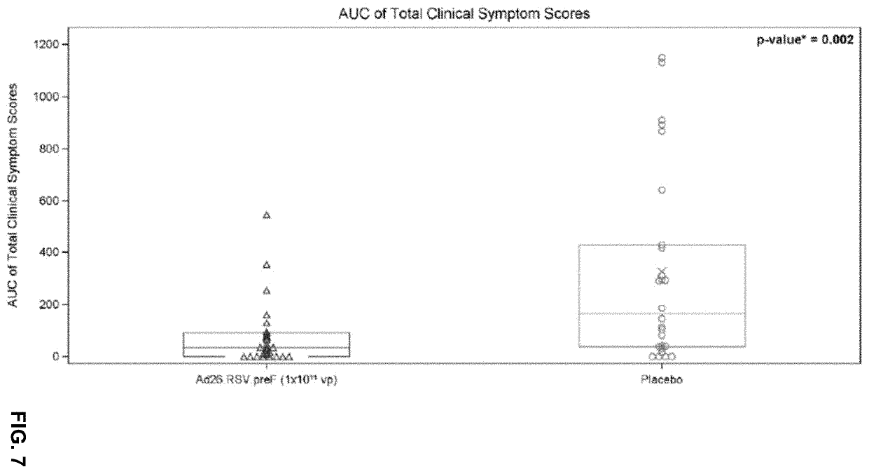

The total clinical symptoms scores, by day, are summarized in FIG. 6, and the AUC of those scores collected from challenge until discharge is depicted in FIG. 7. The median AUC of the total clinical symptoms scores from baseline to discharge was 35 for the Ad26.RSV.preF group and 167 for the Placebo group. The total symptom scores peaked in the afternoon of Day 6 for the placebo group.

3d. Proportion of subjects with symptomatic RSV infection: The percentage of subjects with symptomatic RSV infection was defined in the following ways:
Conservative: the subject has two or more quantifiable RT-PCR measurements on different samples and the subject has one of the following:
   symptoms from two different categories (Upper Respiratory, Lower Respiratory, Systemic, see section 3c) from the Subject Symptoms Card, regardless of grade and assessment timepoint OR
   any Grade 2 symptom from any category.
Liberal (RT-PCR): two or more quantifiable RT-PCR measurements plus any clinical symptom of any severity from the Subject Symptoms Card.

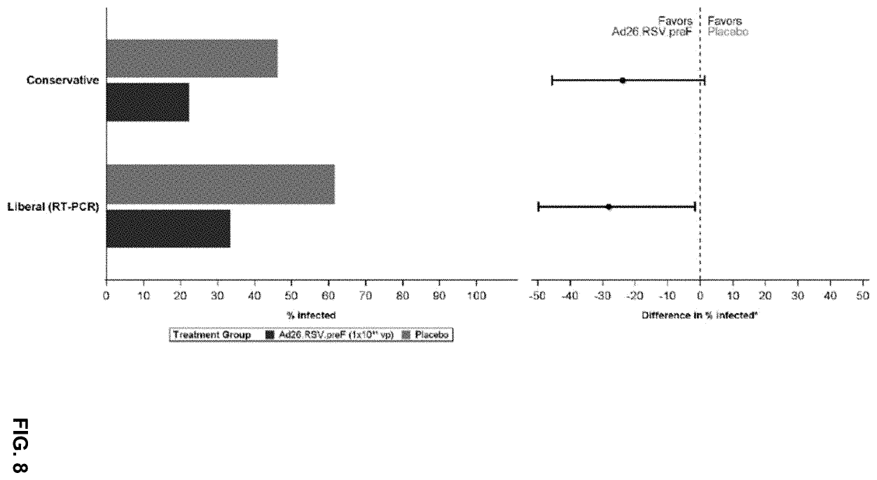

The percentage of subjects with symptomatic RSV infection according to the conservative and liberal definitions is depicted in FIG. 8. Based on the conservative definition, 6/27 (22.2%) subjects were considered infected for the Ad26.RSV.preF group, and 12/26 (46.2%) for the Placebo group, leading to a vaccine efficacy of 51.9% with corresponding 95% CI (−7.4%, 83.2%). Based on the liberal definition, 9/27 (33.3%) subjects were considered infected for the Ad26.RSV.preF group, and 16/26 (61.5%) subjects were considered infected for the Placebo group, leading to a vaccine efficacy of 45.8% with corresponding 95% CI (−1%, 73.8%).

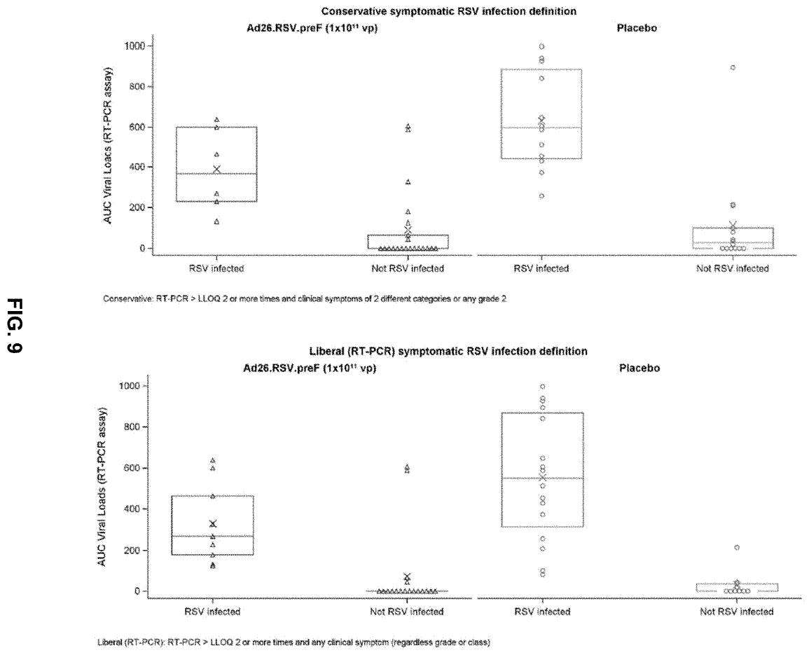

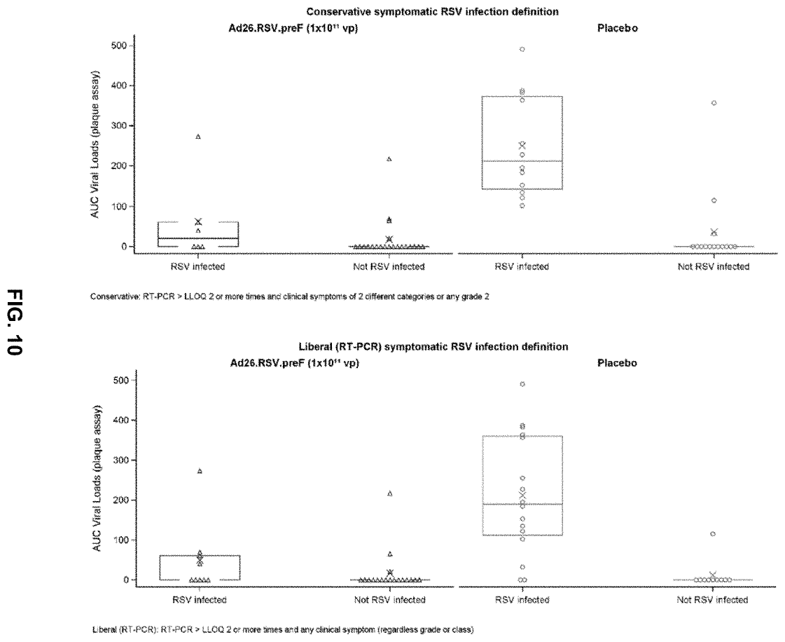

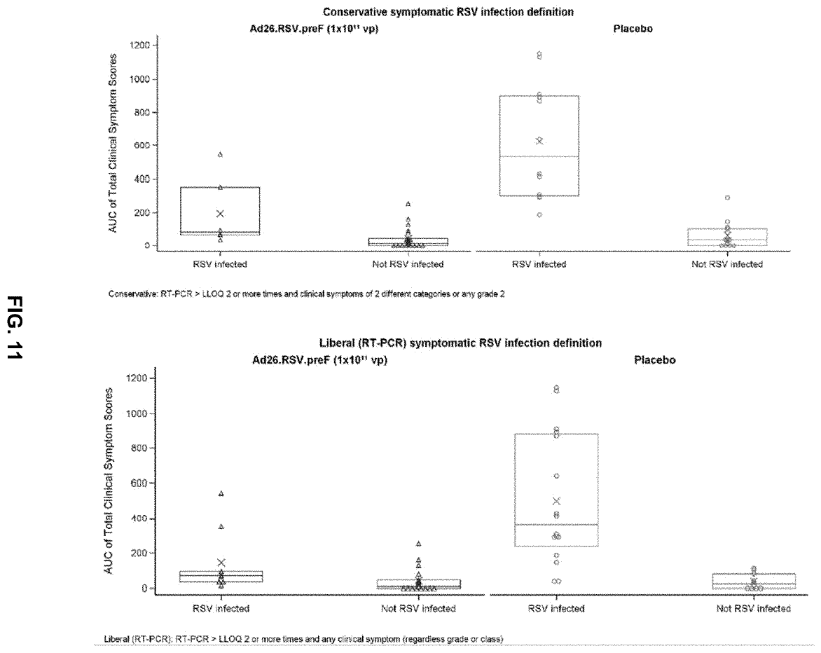

The primary efficacy endpoint, AUC VL determined by RT-PCR of nasal wash samples, is summarized based on the symptomatic RSV infection definitions in FIG. 9. The AUC VL determined by quantitative culture of RSV of nasal wash and the AUC of the total symptom scores are summarized based on the symptomatic RSV infection definitions in FIG. 10 and FIG. 11, respectively.

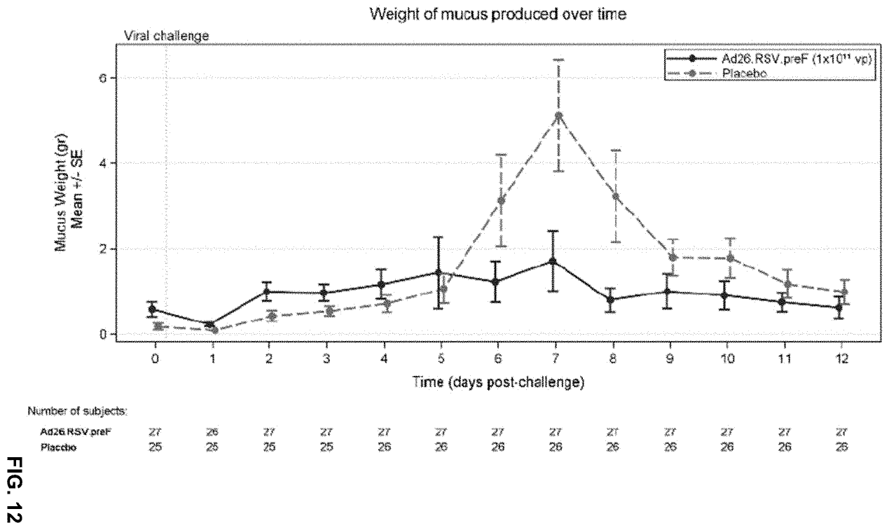

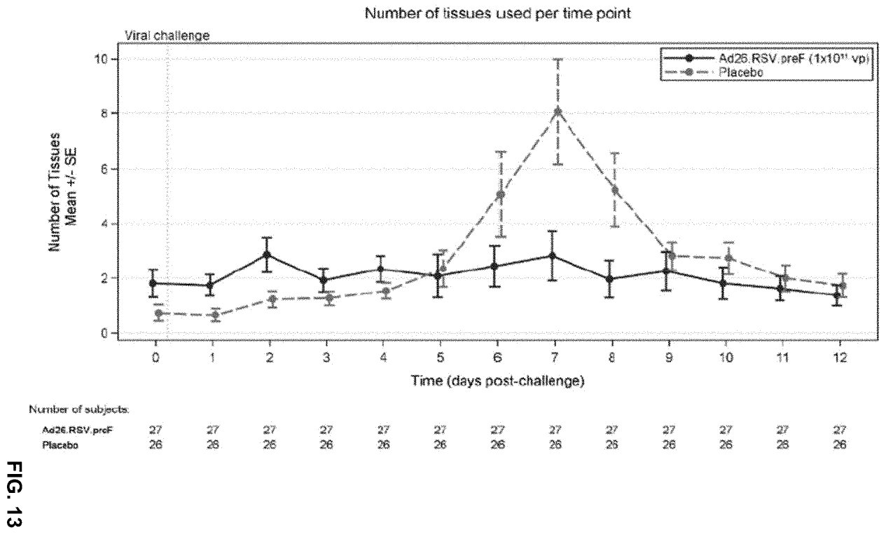

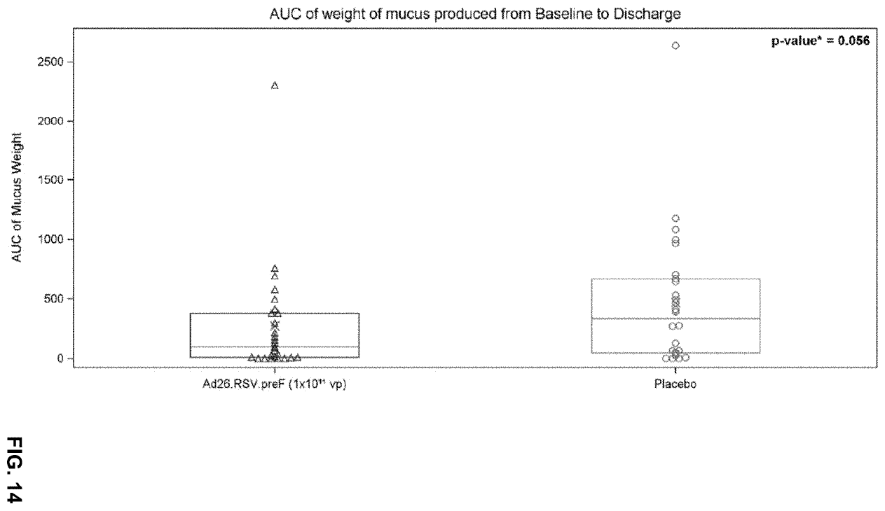

3e. Weight of mucus and number of tissues: The weight of mucus and the number of tissues analyzed for weight of mucus is summarized with the mean and SE, by day in FIG. 12 and FIG. 13 respectively. The peak for both was observed at Day 7. The median AUC of the mucus weight from baseline to discharge was 102 for the Ad26.RSV.preF group and 333 for the Placebo group, as shown in FIG. 14.

4. Immunogenicity endpoints: The immunogenicity analysis was based on the Per-protocol Immunogenicity (PPI) set which contained 61 subjects that were randomized and vaccinated, from whom immunogenicity data were available.

For the primary analysis viral neutralizing antibody against RSV A2 (VNA A2) and Pre-F ELISA were analyzed. Additional data, such as Post F ELISA, VNA RSV A Memphis 37b and Ad26 VNA were also analyzed.

The immunogenicity analysis was carried out using two timepoints: Baseline (vaccination) and 28 days post-vaccination, which included all assessments taken between 22 and 33 days after vaccination.

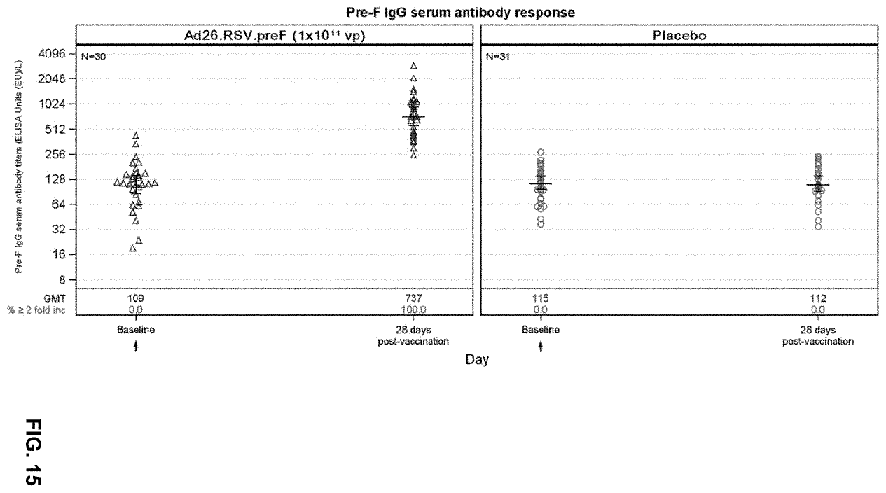

The Pre-F IgG serum antibody response, as assessed by ELISA, is shown in FIG. 15. The geometric mean ratio between 28 days post vaccination and baseline (with 95% CI) of Pre-F ELISA were 6.9 (5.1; 9.4) and 1 (0.9; 1) ELISA units for the Ad26.RSV.preF and Placebo group, respectively.

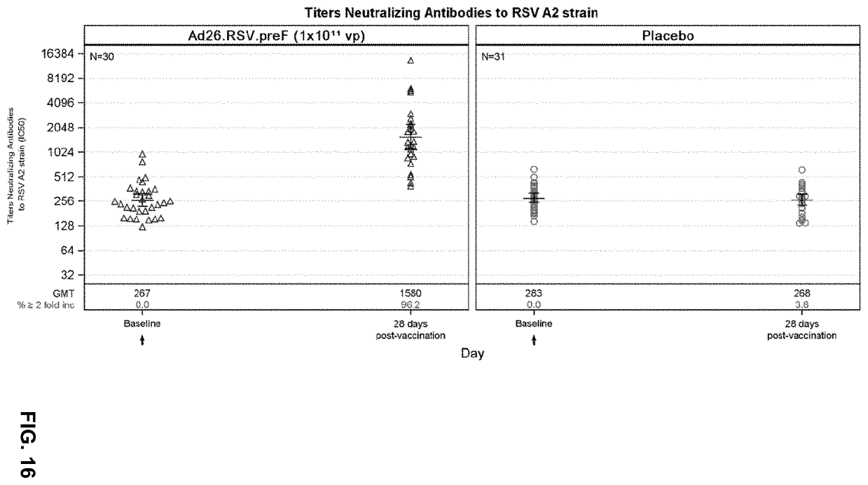

Titers of neutralizing antibodies to RSV A2 strain are shown in FIG. 16. The geometric mean increase and 95% CI of VNA A2 were 5.9 (4.4; 8) and 0.9 (0.8; 1) for the Ad26.RSV.preF and Placebo group, respectively.

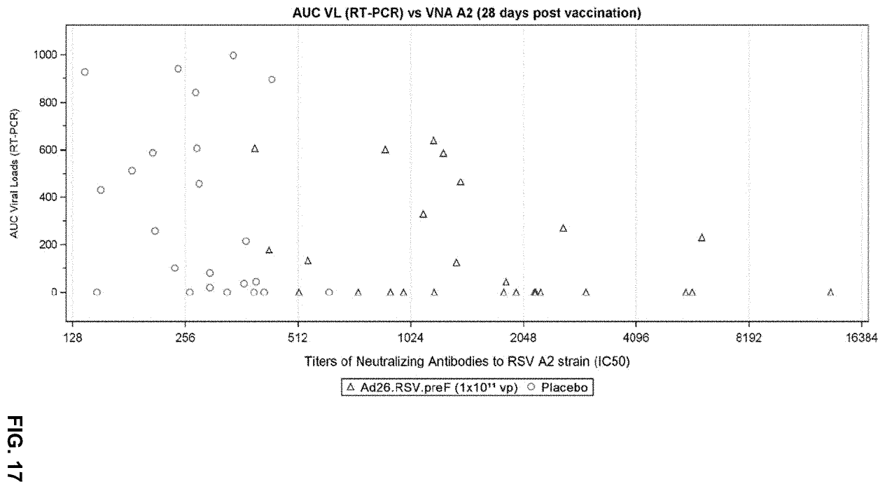

The AUC Viral Load determined by quantitative RT-PCR of nasal wash samples versus 28 days post vaccination VNA A2 responses are plotted in FIG. 17. A similar relationship was observed between AUC VL and the rest of the humoral assays, as well as between AUC of the remaining efficacy endpoints versus the humoral assays.

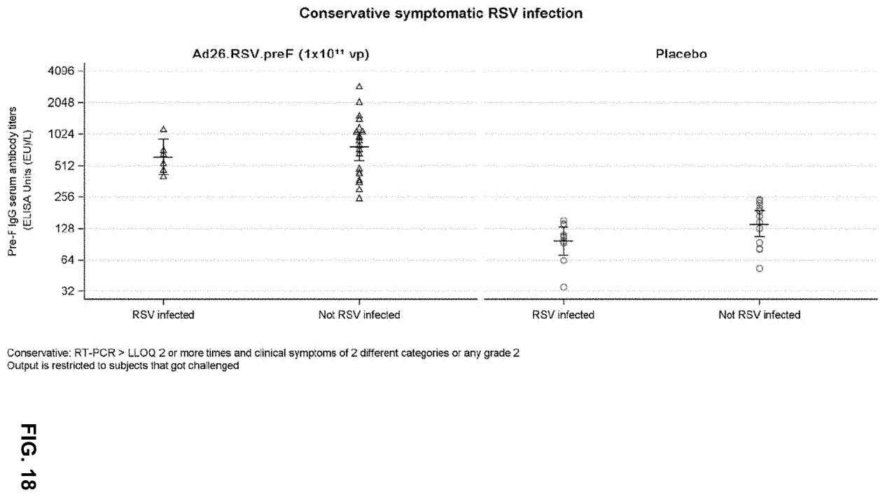

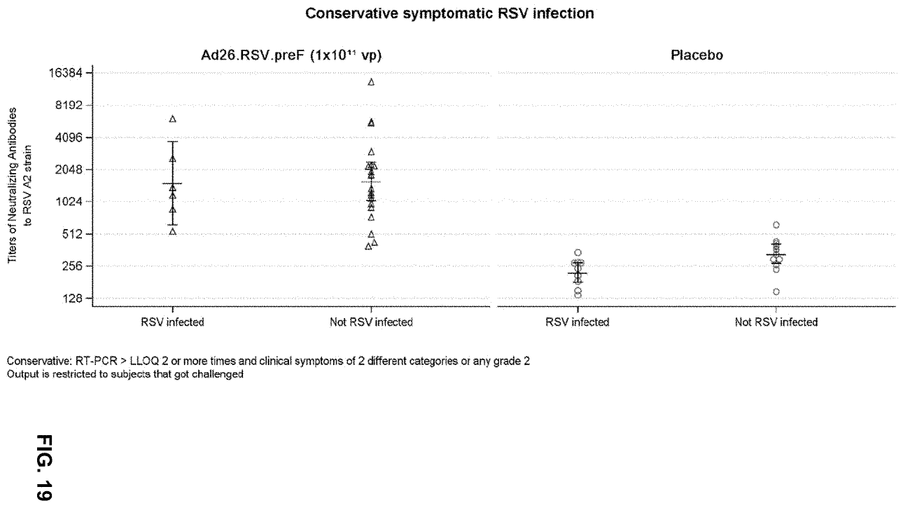

For the conservative symptomatic RSV infection definition, 28 days post vaccination humoral values are presented in FIGS. 18 and 19.

5. Safety: No SAEs were reported. One subject in the active group reported an AE that led to delay of the challenge (Grade 2 Urinary tract infection, not related). One subject in the placebo group reported AEs that led to cancelation of the challenge (Grade 1 Malaise and grade 1 oropharyngeal pain, both not related). The latter subject was afterwards lost to follow-up.

All unsolicited AEs post-vaccination or post-challenge were grade 1 or 2. All solicited local AEs were grade 1 or 2. The most frequently reported solicited local AEs were pain/tenderness and swelling induration, respectively reported in all subjects (100%) and 29.0% of the subjects in the active group and in 18.8% and 3.1% of the subjects in the placebo group. The median time to onset in the active group was 1 day for pain/tenderness and 2 days for swelling induration. The median duration in the active group was 4 and 2 days respectively. Three subjects in the active group and 1 subject in the placebo group reported at least one grade 3 solicited systemic AE. All other solicited systemic AEs were grade 1 or 2. The 3 most frequently reported solicited systemic AEs were Myalgia, Fatigue and Headache. These were reported respectively in 90.3%, 83.9% and 83.9% of the subjects in the active group and in 12.5%, 37.5% and 25.0% of subjects in the placebo group. The median time to onset and duration these solicited systemic AEs was 2 days.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

SEQUENCES
(RSV F protein A2 full length sequence)
SEQ ID NO: 1
MELLILKANAITTILTAVTFCFASGQNITEEFYQSTCSAVSKGYLSALRT
GWYTSVITIELSNIKKNKCNGTDAKIKLIKQELDKYKNAVTELQLLMQST
PATNNRARRELPRFMNYTLNNAKKTNVTLSKKRKRRFLGFLLGVGSAIAS
GVAVSKVLHLEGEVNKIKSALLSTNKAVVSLSNGVSVLTSKVLDLKNYID
KQLLPIVNKQSCSISNIETVIEFQQKNNRLLEITREFSVNAGVTTPVSTY
MLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVS
FFPQAETCKVQSNRVFCDTMNSLTLPSEVNLCNVDIFNPKYDCKIMTSKT
DVSSSVITSLGAIVSCYGKTKCTASNKNRGIIKTFSNGCDYVSNKGVDTV
SVGNTLYYVNKQEGKSLYVKGEPIINFYDPLVFPSDEFDASISQVNEKIN
QSLAFIRKSDELLHNVNAVKSTTNIMITTIIIVIIVILLSLIAVGLLLYC
KARSTPVTLSKDQLSGINNIAFSN
(Trimerization domain)
SEQ ID NO: 2
GYIPEAPRDGQAYVRKDGEWVLLSTFL
(Linker)
SEQ ID NO: 3
SAIG
(RSV preF2.1)
SEQ ID NO: 4
MELLILKANAITTILTAVTFCFASGQNITEEFYQSTCSAVSKGYLGALRT
GWYTSVITIELSNIKEIKCNGTDAKVKLIKQELDKYKNAVTELQLLMQST
PATNNRARRELPRFMNYTLNNAKKTNVTLSKKRKRRFLGFLLGVGSAIAS
GVAVSKVLHLEGEVNKIKSALLSTNKAVVSLSNGVSVLTSKVLDLKNYID
KQLLPIVNKQSCSIPNIETVIEFQQKNNRLLEITREFSVNAGVTTPVSTY
MLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVS
FFPQAETCKVQSNRVFCDTMNSLTLPSEVNLCNVDIFNPKYDCKIMTSKT
DVSSSVITSLGAIVSCYGKTKCTASNKNRGIIKTFSNGCDYVSNKGVDTV
SVGNTLYYVNKQEGKSLYVKGEPIINFYDPLVFPSDEFDASISQVNEKIN
QSLAFIRKSDELLHNVNAVKSTTNIMITTIIIVIIVILLSLIAVGLLLYC
KARSTPVTLSKDQLSGINNIAFSN
(RSV preF2.2)
SEQ ID NO: 5
MELLILKANAITTILTAVTFCFASGQNITEEFYQSTCSAVSKGYLSALRT
GWYTSVITIELSNIKEiKCNGTDAKVKLIKQELDKYKNAVTELQLLMQST
PATNNRARRELPRFMNYTLNNAKKTNVTLSKKRKRRFLGFLLGVGSAIAS
GVAVSKVLHLEGEVNKIKSALLSTNKAVVSLSNGVSVLTSKVLDLKNYID
KQLLPIVNKQSCSIPNIETVIEFQQKNNRLLEITREFSVNAGVTTPVSTY
MLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVS
FFPQAETCKVQSNRVFCDTMNSLTLPSEVNLCNVDIFNPKYDCKIMTSKT
DVSSSVITSLGAIVSCYGKTKCTASNKNRGIIKTFSNGCDYVSNKGVDTV
SVGNTLYYVNKQEGKSLYVKGEPIINFYDPLVFPSNEFDASISQVNEKIN
QSLAFIRKSDELLHNVNAVKSTTNIMITTIIIVIIVILLSLIAVGLLLYC
KARSTPVTLSKDQLSGINNIAFSN
(RSV F pre-F2.1)
SEQ ID NO: 6
ATGGAGCTGCTGATCCTGAAGGCCAACGCCATCACCACCATCCTGACCGC
CGTGACCTTCTGCTTCGCCAGCGGCCAGAACATCACCGAGGAGTTCTACC
AGAGCACCTGCAGCGCCGTGAGCAAGGGCTACCTGGGCGCCCTGAGAACC
GGCTGGTACACCAGCGTGATCACCATCGAGCTGAGCAACATCAAGGAGAT
CAAGTGCAACGGCACCGACGCCAAGGTGAAGCTGATCAAGCAGGAGCTGG
ACAAGTACAAGAACGCCGTGACCGAGCTGCAGCTGCTGATGCAGAGCACC
CCCGCCACCAACAACAGAGCCAGAAGAGAGCTGCCCAGATTCATGAACTA
CACCCTGAACAACGCCAAGAAGACCAACGTGACCCTGAGCAAGAAGAGAA
AGAGAAGATTCCTGGGCTTCCTGCTGGGCGTGGGCAGCGCCATCGCCAGC
GGCGTGGCCGTGAGCAAGGTGCTGCACCTGGAGGGCGAGGTGAACAAGAT
CAAGAGCGCCCTGCTGAGCACCAACAAGGCCGTGGTGAGCCTGAGCAACG
GCGTGAGCGTGCTGACCAGCAAGGTGCTGGACCTGAAGAACTACATCGAC
AAGCAGCTGCTGCCCATCGTGAACAAGCAGAGCTGCAGCATCCCCAACAT
CGAGACCGTGATCGAGTTCCAGCAGAAGAACAACAGACTGCTGGAGATCA
CCAGAGAGTTCAGCGTGAACGCCGGCGTGACCACCCCCGTGAGCACCTAC
ATGCTGACCAACAGCGAGCTGCTGAGCCTGATCAACGACATGCCCATCAC
CAACGACCAGAAGAAGCTGATGAGCAACAACGTGCAGATCGTGAGACAGC
AGAGCTACAGCATCATGAGCATCATCAAGGAGGAGGTGCTGGCCTACGTG
GTGCAGCTGCCCCTGTACGGCGTGATCGACACCCCCTGCTGGAAGCTGCA
CACCAGCCCCCTGTGCACCACCAACACCAAGGAGGGCAGCAACATCTGCC
TGACCAGAACCGACAGAGGCTGGTACTGCGACAACGCCGGCAGCGTGAGC
TTCTTCCCCCAGGCCGAGACCTGCAAGGTGCAGAGCAACAGAGTGTTCTG
CGACACCATGAACAGCCTGACCCTGCCCAGCGAGGTGAACCTGTGCAACG
TGGACATCTTCAACCCCAAGTACGACTGCAAGATCATGACCAGCAAGACC
GACGTGAGCAGCAGCGTGATCACCAGCCTGGGCGCCATCGTGAGCTGCTA
CGGCAAGACCAAGTGCACCGCCAGCAACAAGAACAGAGGCATCATCAAGA
CCTTCAGCAACGGCTGCGACTACGTGAGCAACAAGGGCGTGGACACCGTG
AGCGTGGGCAACACCCTGTACTACGTGAACAAGCAGGAGGGCAAGAGCCT
GTACGTGAAGGGCGAGCCCATCATCAACTTCTACGACCCCCTGGTGTTCC
CCAGCGACGAGTTCGACGCCAGCATCAGCCAGGTGAACGAGAAGATCAAC
CAGAGCCTGGCCTTCATCAGAAAGAGCGACGAGCTGCTGCACAACGTGAA
CGCCGTGAAGAGCACCACCAACATCATGATCACCACCATCATCATCGTGA
TCATCGTGATCCTGCTGAGCCTGATCGCCGTGGGCCTGCTGCTGTACTGC
AAGGCCAGAAGCACCCCCGTGACCCTGAGCAAGGACCAGCTGAGCGGCAT
CAACAACATCGCCTTCAGCAACTGA
(RSV F pre-F2.2)
SEQ ID NO: 7
ATGGAGCTGCTGATCCTGAAGGCCAACGCCATCACCACCATCCTGACCGC
CGTGACCTTCTGCTTCGCCAGCGGCCAGAACATCACCGAGGAGTTCTACC
AGAGCACCTGCAGCGCCGTGAGCAAGGGCTACCTGAGCGCCCTGAGAACC
GGCTGGTACACCAGCGTGATCACCATCGAGCTGAGCAACATCAAGGAGAT
CAAGTGCAACGGCACCGACGCCAAGGTGAAGCTGATCAAGCAGGAGCTGG
ACAAGTACAAGAACGCCGTGACCGAGCTGCAGCTGCTGATGCAGAGCACC
CCCGCCACCAACAACAGAGCCAGAAGAGAGCTGCCCAGATTCATGAACTA
CACCCTGAACAACGCCAAGAAGACCAACGTGACCCTGAGCAAGAAGAGAA
AGAGAAGATTCCTGGGCTTCCTGCTGGGCGTGGGCAGCGCCATCGCCAGC
GGCGTGGCCGTGAGCAAGGTGCTGCACCTGGAGGGCGAGGTGAACAAGAT
CAAGAGCGCCCTGCTGAGCACCAACAAGGCCGTGGTGAGCCTGAGCAACG
GCGTGAGCGTGCTGACCAGCAAGGTGCTGGACCTGAAGAACTACATCGAC
AAGCAGCTGCTGCCCATCGTGAACAAGCAGAGCTGCAGCATCCCCAACAT
CGAGACCGTGATCGAGTTCCAGCAGAAGAACAACAGACTGCTGGAGATCA
CCAGAGAGTTCAGCGTGAACGCCGGCGTGACCACCCCCGTGAGCACCTAC
ATGCTGACCAACAGCGAGCTGCTGAGCCTGATCAACGACATGCCCATCAC
CAACGACCAGAAGAAGCTGATGAGCAACAACGTGCAGATCGTGAGACAGC
AGAGCTACAGCATCATGAGCATCATCAAGGAGGAGGTGCTGGCCTACGTG
GTGCAGCTGCCCCTGTACGGCGTGATCGACACCCCCTGCTGGAAGCTGCA
CACCAGCCCCCTGTGCACCACCAACACCAAGGAGGGCAGCAACATCTGCC
TGACCAGAACCGACAGAGGCTGGTACTGCGACAACGCCGGCAGCGTGAGC
TTCTTCCCCCAGGCCGAGACCTGCAAGGTGCAGAGCAACAGAGTGTTCTG
CGACACCATGAACAGCCTGACCCTGCCCAGCGAGGTGAACCTGTGCAACG
TGGACATCTTCAACCCCAAGTACGACTGCAAGATCATGACCAGCAAGACC
GACGTGAGCAGCAGCGTGATCACCAGCCTGGGCGCCATCGTGAGCTGCTA
CGGCAAGACCAAGTGCACCGCCAGCAACAAGAACAGAGGCATCATCAAGA
CCTTCAGCAACGGCTGCGACTACGTGAGCAACAAGGGCGTGGACACCGTG
AGCGTGGGCAACACCCTGTACTACGTGAACAAGCAGGAGGGCAAGAGCCT
GTACGTGAAGGGCGAGCCCATCATCAACTTCTACGACCCCCTGGTGTTCC
CCAGCAACGAGTTCGACGCCAGCATCAGCCAGGTGAACGAGAAGATCAAC
CAGAGCCTGGCCTTCATCAGAAAGAGCGACGAGCTGCTGCACAACGTGAA
CGCCGTGAAGAGCACCACCAACATCATGATCACCACCATCATCATCGTGA
TCATCGTGATCCTGCTGAGCCTGATCGCCGTGGGCCTGCTGCTGTACTGC
AAGGCCAGAAGCACCCCCGTGACCCTGAGCAAGGACCAGCTGAGCGGCAT
CAACAACATCGCCTTCAGCAACTGA

1. A method of inducing a protective immune response against respiratory syncytial virus (RSV) infection in a human subject in need thereof, comprising intramuscularly administering to the subject an effective amount of a pharmaceutical composition, a vaccine, comprising an adenoviral vector comprising a nucleic acid encoding an RSV F polypeptide that is stabilized in a pre-fusion conformation, wherein the effective amount of the pharmaceutical composition comprises about 1×10¹⁰ to about 1×10¹² viral particles of the adenoviral vector per dose.

2. The method of claim 1, wherein the adenoviral vector is replication-incompetent and has a deletion in at least one of the adenoviral early region 1 (E1 region) and the early region 3 (E3 region).

3. The method of claim 2, wherein the adenoviral vector is a replication-incompetent Ad26 adenoviral vector having a deletion of the E1 region and the E3 region.

4. The method of claim 2, wherein the adenoviral vector is a replication-incompetent Ad35 adenoviral vector having a deletion of the E1 region and the E3 region.

5. The method of claim 1, wherein the recombinant RSV F polypeptide encoded by the adenoviral vector has the amino acid sequence of SEQ ID NO: 4 or SEQ ID NO: 5.

6. The method of claim 1, wherein the nucleic acid encoding the RSV F polypeptide comprises the polynucleotide sequence of SEQ ID NO: 6 or SEQ ID NO: 7.

7. The method of claim 1, wherein the effective amount of the pharmaceutical composition comprises about 1×10¹¹ viral particles of the adenoviral vector per dose.

8. The method of claim 1, further comprising administering to the subject an effective amount of the pharmaceutical composition comprising about 1×10¹⁰ to about 1×10¹² viral particles of the adenoviral vector per dose after the initial administration.

9. The method of claim 1, wherein the subject is susceptible to the RSV infection.

10. The method of claim 1, wherein the protective immune response is characterized by an absent or reduced RSV viral load in the nasal track and/or lungs of the subject upon exposure to RSV.

11. The method of claim 1, wherein the protective immune response is characterized by an absent or reduced RSV clinical symptom in the subject upon exposure to RSV.

12. The method of claim 1, wherein the protective immune response is characterized by the presence of neutralizing antibodies to RSV and/or protective immunity against RSV, detected 8 to 35 days after administration of the pharmaceutical composition.

13. The method of claim 1, wherein the administration does not induce any severe adverse event.

14. A method of preventing infection and/or replication of RSV without inducing a severe adverse effect in a human subject in need thereof, comprising prophylactically administering intramuscularly to the subject an effective amount of a pharmaceutical composition, a vaccine, comprising about 1×10¹⁰ to about 1×10¹² viral particles per dose of an adenoviral vector comprising a nucleic acid encoding an RSV F polypeptide having the amino acid sequence of SEQ ID NO: 4 or SEQ ID NO: 5, wherein the adenoviral vector is replication-incompetent.

15. The method of claim 14, wherein the adenoviral vector is a replication-incompetent Ad26 adenoviral vector having a deletion of the E1 region and the E3 region.

16. The method of claim 14, wherein the nucleic acid encoding the RSV F polypeptide comprises the polynucleotide sequence of SEQ ID NO: 6 or SEQ ID NO: 7.

17. The method of claim 14, wherein the effective amount of the pharmaceutical composition comprises about 1×10¹¹ viral particles of the adenoviral vector per dose.

18. The method of claim 14, further comprising administering to the subject an amount of the pharmaceutical composition comprising about 1×10¹⁰ to about 1×10¹² viral particles of the adenoviral vector per dose after the initial administration.

19. The method of claim 14, wherein the subject is susceptible to the RSV infection.

20. The method of claim 14, wherein the prevented infection and/or replication of RSV is characterized by an absent or reduced RSV viral load in the nasal track and/or lungs of the subject.

21. The method of claim 14, wherein the prevented infection and/or replication of RSV is characterized by an absent or reduced RSV clinical symptom in the subject upon exposure to RSV.

22. The method of claim 14, wherein the protective immune response is characterized by the presence of neutralizing antibodies to RSV and/or protective immunity against RSV, detected 8 to 35 days after administration of the pharmaceutical composition.
